(19) United States
(12) Patent Application Publication
(10) Pub. No.: US 2016/0300038 A1
(43) Pub. Date: Oct. 13, 2016

(54) METHODS OF NORMALIZING MEASURED DRUG CONCENTRATIONS IN URINE USING PATIENT SPECIFIC DATA AND TESTING FOR POTENTIAL NON-COMPLIANCE WITH A CHRONIC DRUG TREATMENT REGIMEN

(71) Applicant: Ameritox, Ltd., Baltimore, MD (US)

(72) Inventors: Gregory L. McIntire, Greensboro, NC (US); Ayodele Morris, Midland, TX (US); Oneka Cummings, Baltimore, MD (US)

(21) Appl. No.: 15/097,359

(22) Filed: Apr. 13, 2016

Related U.S. Application Data
(60) Provisional application No. 62/146,806, filed on Apr. 13, 2015.

(51) Int. Cl. G06F 19/00; G01N 33/94

(52) U.S. Cl. CPC G06F 19/3456; G01N 33/9486; G01N 33/948

(57) ABSTRACT

Methods for monitoring subject compliance with a prescribed treatment regimen are disclosed. In an embodiment, the method comprises measuring a drug or metabolite level in urine of a subject and normalizing the measured drug or inverse of the metabolite level as a function of one or more parameters associated with the subject followed by transformation of the normalized data using the natural log of the normalized data. Embodiments of the methods use patient derived parameters together with the prescribed dose to affect a normalized and transformed value that can be compared to a normalized and transformed standard distribution derived from a body of collected urine fluid test results.

### PRIORITY

This application claims priority to U.S. provisional application Ser. No. 62/146,806 filed on Apr. 13, 2015, the entirety of which is incorporated herein.

### TECHNICAL FIELD

The present disclosure provides methods for detecting and quantifying a subject's drug use by, inter alia, testing a biological sample from said subject consisting of urine and relating it to a standard population of drug results from known patients prescribed the drug and testing positive for that drug within the limits of the analytical method, e.g., LC/MSMS.

### BACKGROUND

Drug testing in biological fluid samples is well accepted for monitoring both legitimate and illegitimate drug use in populations of chronic pain patients, substance abuse patients, and Mental Health Patients. For example, pain patients who are prescribed chronic opioid therapy (COT) should be tested at least twice per year to determine if a) they are taking their medication at all, b) they are taking additional prescription medications (unknown to the prescribing physician), or c) they are taking illicit drugs in addition to the prescribed opiate/opioid (Cuoto, J. E., et. al.). While positive test results can be informative, comparison to transformed and normalized data for a large population of patients can be especially useful in determining if an individual patient is consistent with that population or outside a reasonable variation from the mean of that population.

Most often, the historical distribution is transformed and normalized to be consistent with a Gaussian distribution. Gaussian distributions are symmetric with uniform variation (e.g., standard deviation (std dev)) in either direction from the mean which for this distribution is also the median of the population. Thus, it is expected that a fixed percentage (68%) of the population of a true Gaussian distribution will be between −1 and +1 std dev units from the mean. An even greater amount of the population (95%) will be between −2 and +2 std dev units from the mean. Conversely, only 5% of the population will lie outside −2 to +2 std dev units from the population mean. Thus, transformation and normalization of an individual patient datum followed by comparison to a historical Gaussian distribution can determine whether this patient is consistent with the population of known patients for the drug they have been prescribed. This alone cannot confirm compliance or noncompliance with a prescribed drug treatment paradigm, but together with clinical observations, traditional compliance tools (i.e., pill counts, prescription refills, interviews, etc) can be used to assess a complete picture of the patient and their drug use.

Methods of the present disclosure are used to create a transformed and normalized historical Gaussian distribution of urine drug data which can accurately identify which patients are within +/−2 std dev units from the mean of that population and thus are likely consistent with that population, i.e., compliant with their medical treatment paradigm. The patient data tested are different from the patient data used to construct the historical Gaussian distribution. This process requires the use of patient derived criteria; both from UDT (e.g, specific gravity, pH, creatinine concentration, etc.) and non-urine derived patient characteristics (e.g, weight, height, age, sex, etc). The process is described through examples including buprenorphine (e.g., SUBUTEX®), alprazolam (Xanax®), hydrocodone (Vicodin), oxycodone (Oxycontin) and oxazepam.

Chronic drug therapy is often prescribed for ongoing diseases, disorders, and for alleviating symptoms thereof. Chronic drug therapy is frequently characterized by an ongoing, regular ingestion of one or more drugs, typically at the direction of a physician (e.g., in accordance with a prescribed therapeutic regimen).

For example, buprenorphine is a semisynthetic opioid partial agonist-antagonist that is indicated for the management of chronic moderate acute pain (e.g. Buprenex) in non-opioid-tolerant individuals in lower dosages (e.g. Butrans®) and to control moderate to severe chronic pain in even smaller doses (e.g. Temgesic, Butrans®) (Baselt, 2004, SUBOXONE® Highlights of Prescribing Information, 2014). It is primarily used to treat opioid addiction (encompassing both heroin abuse and prescription opioid pain medication abuse) in higher dosages, alone (e.g. SUBUTEX®), or in combination with naloxone (e.g. SUBOXONE®), an opioid antagonist (National Drug Intelligence Center, 2004). SUBUTEX® and SUBOXONE® were the first narcotic drugs available under the 2000 Drug Abuse Treatment Act (DATA) for the office-based treatment of opioid dependence, which provided patients better access to treatment (Kacinko, et al.). The opioid partial agonist property makes buprenorphine appealing for opioid addiction/dependence treatment, since alternate options like full agonists (e.g. methadone) can result in dose dependent physical reliance and tolerance. In addition, buprenorphine can produce euphoria, especially if injected. While this and other subjective effects are what help maintain compliance in opioid dependent individuals receiving treatment, at the same time they promote risks of addiction, abuse, misuse and criminal diversion, similar to other opioids, even at recommended doses. The abuse potential, though, is believed to be lower than opioid full agonists. Still, monitoring for diversion is recommended (Clinical Guidelines for the use of buprenorphine in the treatment of opioid addiction, 2004). Variable excretion patterns of buprenorphine have been suggested to indicate metabolic changes requiring dose adjustment (Kacinko et al., 2009). As such periodic assessment to monitor patients for compliance and dose adjustment, while being prescribed a pain regimen, is an important component of their care and is recommended (Suboxone Highlights of Prescribing Information, 2014). Buprenorphine is metabolized to norbuprenorphine by N-dealkylation, and buprenorphine glucuronide and norbuprenorphine glucuronide following phase II metabolism (Cone et al.). It is excreted extensively as the glucuronides in urine (Baselt, 2004).

Because of known dependency risks, subjects on chronic drug (e.g., opioid) therapy regimens are typically screened periodically to monitor compliance and efficacy of the prescribed therapy (Webster, 2013). Due to the limits of known screening techniques, however, subjects misusing the prescribed opioid often pass basic screening tests performed at a clinic and continue to receive the opioid. Furthermore, patients treated with opioids for the management of chronic pain also have been documented to under-report their use of medications. As a result, health care professionals often use external sources of information such as interviews with the subject's spouse and/or friends, review of the subject's medical records, input from prescription monitoring programs, and testing of biological samples (e.g., fluids) to detect misuse of drugs and non-compliance with the prescribed opioid regimen.

Known drug screening methods generally can detect the presence or absence of a drug in a sample. Samples of fluids are generally obtained from the subject, for example, urine, blood, oral fluid or plasma. Such known screening methods do not in and of themselves, however, enable the health care professional reviewing the lab result to determine whether the subject is non-compliant with a prescribed drug regimen; e.g. buprenorphine

While drug concentrations can be discerned in and from urine, the results are not always directly translatable to compliance. Normalized curves derived from carefully controlled, small populations of patients, for a series of drugs have been published for urine drug samples (Couto, et al., 2011; Couto, et al, 2009, Leider, H. 2014) such that a physician can quickly compare the patient's results with data from these normal populations to help decide if the patient is compliant. While others have criticized these works (McCloskey, et al. 2013, McCloskey and Stickle 2013), the curves do have utility in everyday medical practice.

Others have proposed using large data sets to predict “normal” population data to help physicians determine if patients are compliant. However, these data are often skewed to higher or lower concentrations and thus do not afford a quick or easy assessment of individual patient data. These minor transformations do not meet the strict criteria of a Gaussian distribution. For example, the provision of a “standard curve” derived from measured concentrations of drug from a population of patient results has been proposed to include normalization of these data via division of the concentration values by the patient's creatinine level and subsequent transformation of the normalized values by a natural log transformation. It is generally agreed upon that creatinine levels reflect the level of “hydration” of the patient which is reflected in the concentration of drug determined in the urine. For example, two patients with identical demographics taking the same dose of drug per day, would be expected to have different drug concentrations depending upon their relative levels of hydration. Yet, the resulting “standard curves” from this simple approach are often skewed and hence do not offer statistical assessment of the individual patient data to be referred to the curve. Clearly, this model of drug concentrations with dose is insufficient to describe the distribution of drug over all normal patients (i.e., compliant patients).

### SUMMARY

The present disclosure provides methods for monitoring patient adherence to chronic drug therapy, for example as a component of treating a subject for a chronic condition such as pain or opioid dependence.

In various embodiments, the present disclosure provides methods for detecting or monitoring a subject's potential non-compliance with a prescribed drug regimen (e.g., a buprenorphine drug regimen, etc.) based at least in part on patient-specific data. In various embodiments, the prescribed drug regiment comprises chronic administration of opiates, opioids, benzodiazepines, muscle relaxants (e.g., carisoprodol), antipsychotics, antidepressants, cardiovascular drugs, non-opioid analgesics, antihistamines, sedative hypnotics (i.e., ambien, lunesta), anticonvulsants (i.e., tegretol, Keppra, etc), barbiturates, buprenorphine, naloxone, naltrexone, ADHD drugs (adderol, ritalin, strattera), tricyclic antidepressants, etc. First metabolites include oxazepam, norHydrocodone, norOxycodone, 7 amino-clonazepam, alpha-hydroxy alprazolam, EDDP (methadone metabolite), OPC 3373 (aripiprazole metabolite), dehydroaripiprazole, hydroxyquetiapine, carboxyquetiapine, quetiapine sulfoxide, hydroxyrespiradol, hydroxyduloxetine, norfluoxetine, or any other chronically-administered drug, as easily discovered in texts such as “Disposition of Toxic Drugs and Chemicals in Man” Baselt, 10^{th} ed, Biomedical Publications, Seal Beach, Calif. In an embodiment, the method can identify a subject at risk of drug misuse. In some embodiments, the method provides assistance in reducing the risk of drug misuse in a subject further comprising reducing a prescribed daily dose of a drug (e.g., a buprenorphine drug) for the subject and/or counseling the subject if the drug concentration in urine of the subject falls outside the upper confidence interval or outside of the upper limit of the mathematically normalized and transformed concentration range for the daily dose of the drug (e.g., if the subject is identified as potentially non-compliant with the prescribed drug regimen, e.g. buprenorphine). In some embodiments, the method can identify the risk of drug misuse (e.g., buprenorphine) in a subject further comprising counseling the subject if the drug concentration in urine of the subject falls outside the lower confidence interval or outside of the lower limit of the mathematically normalized and transformed concentration range for the daily dose of the drug (e.g., if the subject is identified as potentially non-compliant with the prescribed drug regimen). These and other embodiments can comprise performing mathematical normalization and transformation to yield a normalized and transformed drug concentration determined from a urine sample from a subject and comparing that mathematically normalized and transformed drug concentration to a Gaussian distribution curve prepared from a body of known test subjects who were both prescribed the drug of interest (e.g., a chronically administered drug) and tested positive for the drug and/or metabolite in provided urine. While additional criteria can be applied to the exclusion/acceptance of historical data from this Gaussian distribution, such as a requirement for repeat testing, or acceptable sample validity testing results, to date, these other characteristics have not had a significant impact on the nature of these distributions.

Embodiments of the present disclosure include methods for identifying samples in the lower and upper extremes of a mathematically normalized and transformed distribution relevant to that drug. For example, methods of the present disclosure comprise identifying samples in the lower 2.5% and the upper 2.5% extremes of the mathematically normalized and transformed Gaussian distribution of a specific drug (e.g., the drug itself and/or a metabolite thereof) concentration in urine. Furthermore, relative to known methods, methods of the present disclosure can differentiate between compliance and non-compliance for patients providing urine samples for testing. Finally, relative to known methods, methods of the present disclosure can differentiate between compliance and non-compliance for patients who would appear to be beyond the 2.5% cutoff using other methods.

In another embodiment, a method of the present disclosure uses a body of collected test results of urine samples for the drug or drug metabolite of interest to form a mathematically normalized and transformed database. As opposed to standard curves where carefully controlled, relatively small data sets (i.e., prospective clinical trials), are used to construct “normal” curves for comparison to current drug testing results, the present methods make use of data obtained for the drug or metabolite of the drug of interest and the accompanying demographics and dose data to construct a mathematically normalized and transformed standard curve for urine testing results regardless of dose, time of sample donation, time of dosing, and concurrent medications (if any). Thus, the samples used for this mathematically normalized and transformed standard curve may include samples from subjects identified as high or low metabolizers, subjects with impaired kidney or liver function, subjects using drugs with overlapping metabolites on the same day, and/or subjects taking medication on an inconsistent schedule. However, this process does exclude samples without a discrete value for the drug concentration in question (i.e., >twice the Upper Limit of Linearity (ULOL) or <Lower Limit of Quantitation (LOQ)), and samples that might have been positive for the drug of interest but that were not prescribed that drug. Additional criteria can be applied to the exclusion/acceptance of historical data from this mathematically normalized and transformed Gaussian distribution, such as a requirement for repeat testing, or acceptable sample validity testing results, to date, these other characteristics have not had a significant impact on the nature of these distributions.

This top-down approach to preparing a mathematically normalized and transformed standard curve for urine derived samples provides a reliable comparison of mathematically normalized and transformed urine derived drug (e.g., buprenorphine) concentrations to an overall population comprised of at least 500 data points, more preferably at least 1000 data points, and most preferably at least 5000 data points.

In other embodiments, both primary and secondary metabolites are measured allowing the use of a ratio of metabolite 1 to metabolite 2 or vice versa. It is envisioned that metabolite 1 may be the parent drug originally dosed to the patient (e.g., buprenorphine). In some embodiments, metabolite 2 is a metabolite of buprenorphine, such as norbuprenorphine, buprenorphine glucuronide and/or norbuprenorphine glucuronide. This approach towards establishing historical distributions of drugs and metabolites from urine drug testing has also been demonstrated for hydrocodone and norhydrocodone and oxycodone and noroxycodone. This application makes use of and claims all the properties in their entirety of U.S. Provisional Patent Application Ser. No. 62/035,821, wherein the use of a ratio of metabolite 1 to metabolite 2 or vice versa to be the focus of a mathematical transformation to create a normalized distribution is described. It is further recognized that if a ratio of drug concentrations is used in this embodiment, that “normalization” is not required inasmuch as mathematically, the normalization of each value cancels out in the ratio approach.

These embodiments and other embodiments of the present disclosure are described in further detail below.

### BRIEF DESCRIPTION OF THE DRAWINGS

FIG. 1 shows a histogram of the buprenorphine drug concentrations observed from a body of collected urine test results used to generate the mathematically normalized and transformed standard curve for Buprenorphine from Urine.

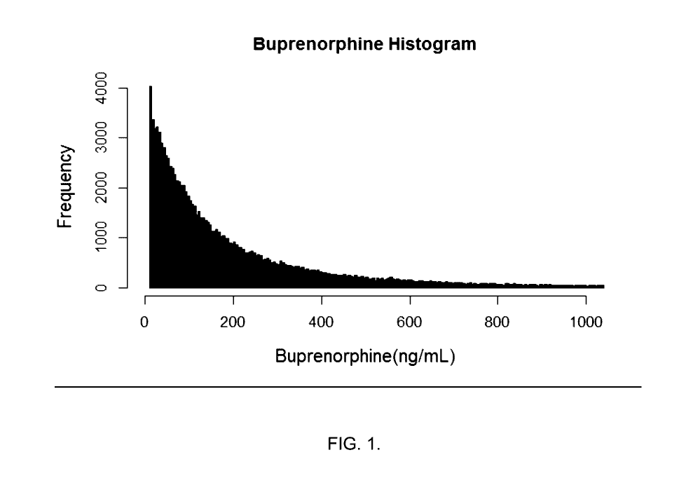

FIG. 2 shows the corresponding kernel density estimation plot derived from the data in FIG. 1.

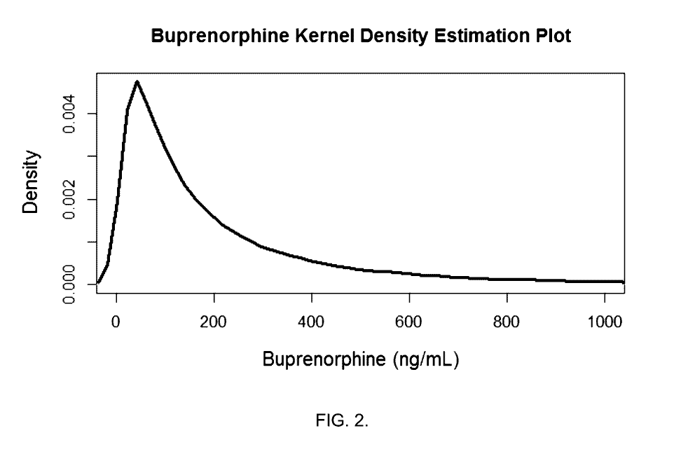

FIG. 3 shows the impact of mathematically normalizing, transforming, and standardizing the data presented in FIG. 1 using subject specific parameters or transformed variables arising from these parameters.

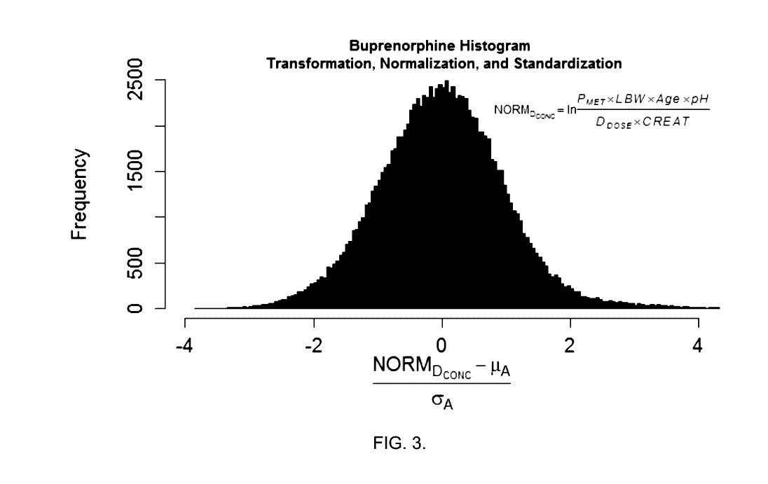

FIG. 4 shows the corresponding kernel density estimation plot derived from the mathematically normalized, transformed, and standardized data presented in FIG. 3.

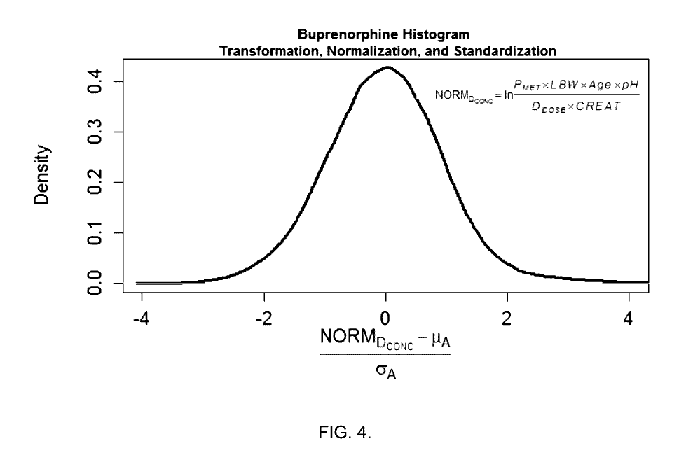

FIG. 5 shows a least squares minimized best fit Gaussian distribution derived from the normalized, transformed, and standardized data from FIG. 1 (i.e., FIG. 4).

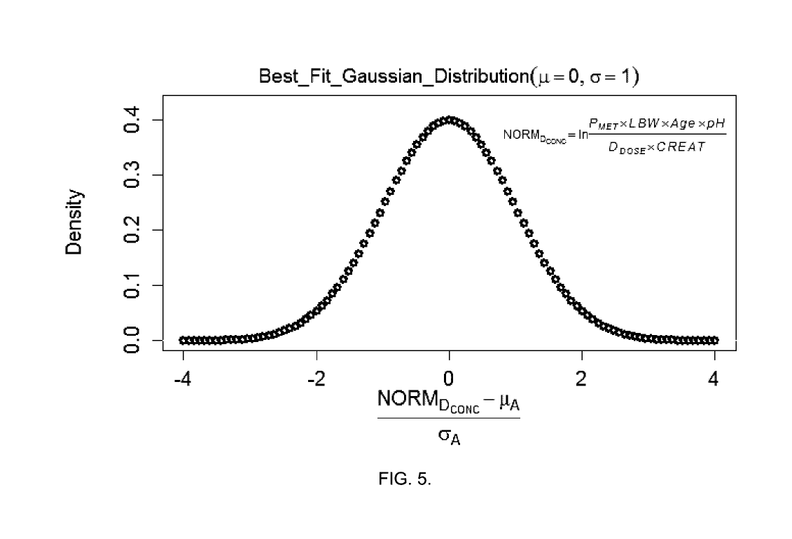

FIG. 6 shows an overlay of the least squares minimized best fit Gaussian distribution (circles) and the kernel density estimation plot (solid line) derived from the normalized, transformed, and standardized data from FIG. 1 (i.e., FIG. 4).

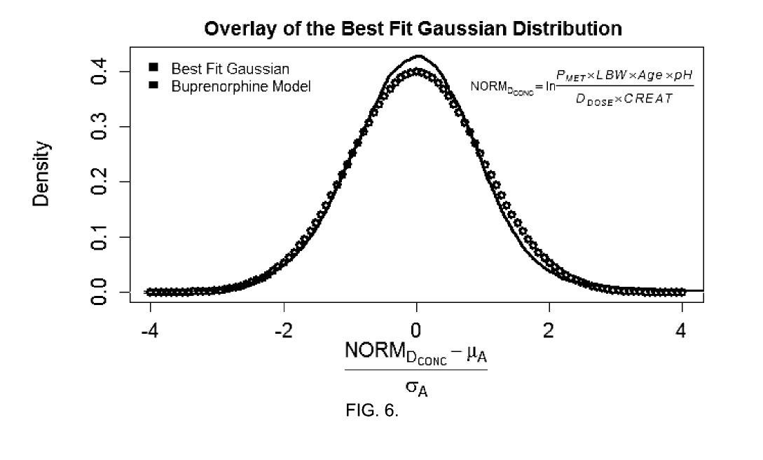

FIG. 7 shows a histogram of the alprazolam drug concentrations observed from a body of collected urine test results used to generate the mathematically normalized and transformed standard curve for alprazolam from Urine.

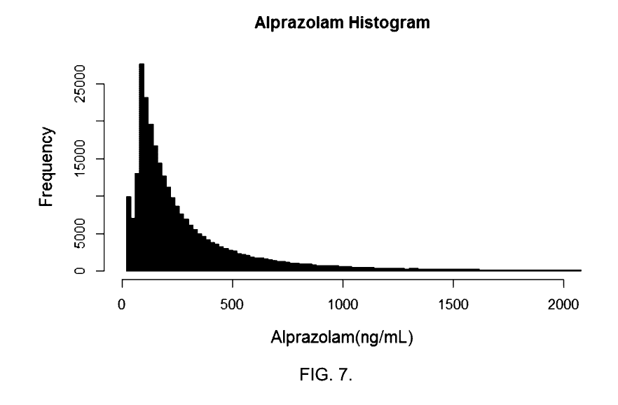

FIG. 8 shows the corresponding kernel density estimation plot derived from the data in FIG. 7.

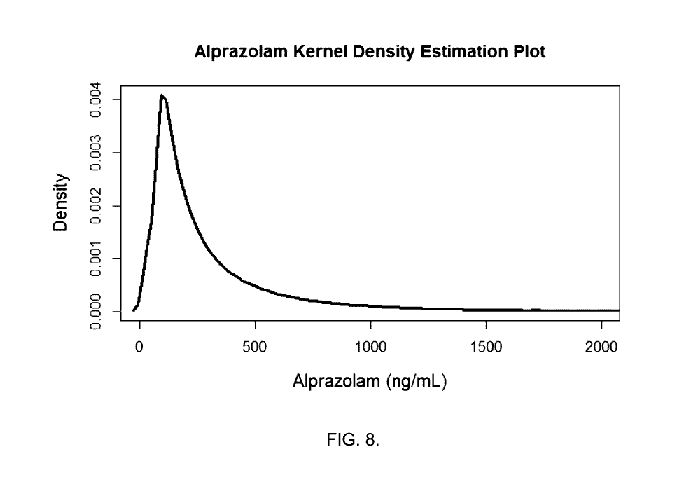

FIG. 9 shows the impact of mathematically normalizing, transforming, and standardizing the data presented in FIG. 7 using subject specific parameters or transformed variables arising from these parameters.

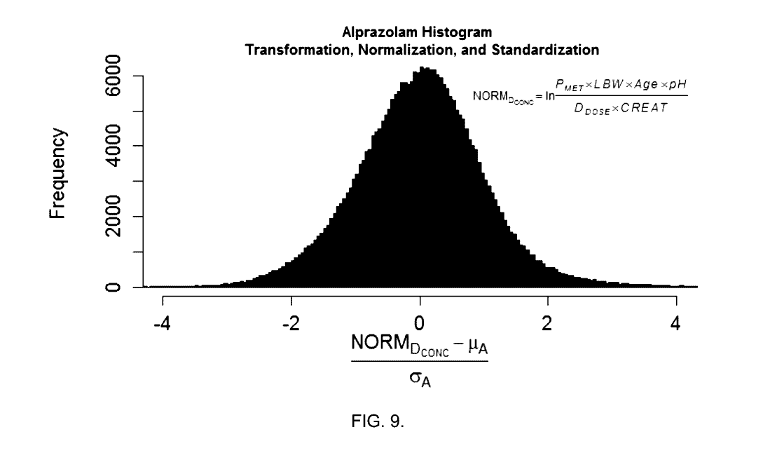

FIG. 10 shows the corresponding kernel density estimation plot derived from the mathematically normalized, transformed, and standardized data presented in FIG. 9.

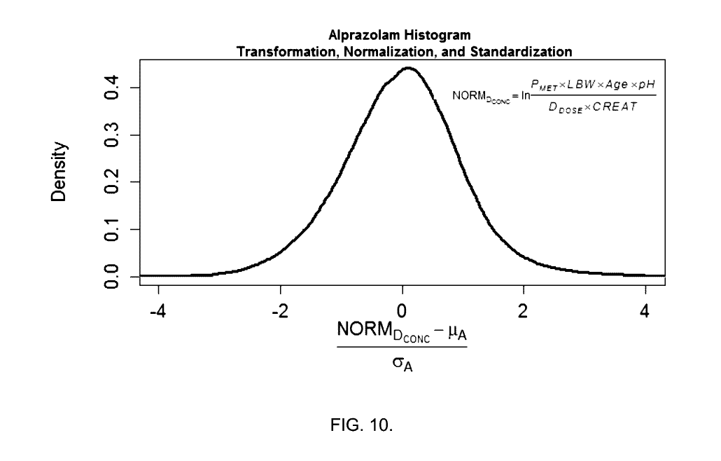

FIG. 11 shows a least squares minimized best fit Gaussian distribution derived from the normalized, transformed, and standardized data from FIG. 7 (i.e., FIG. 10).

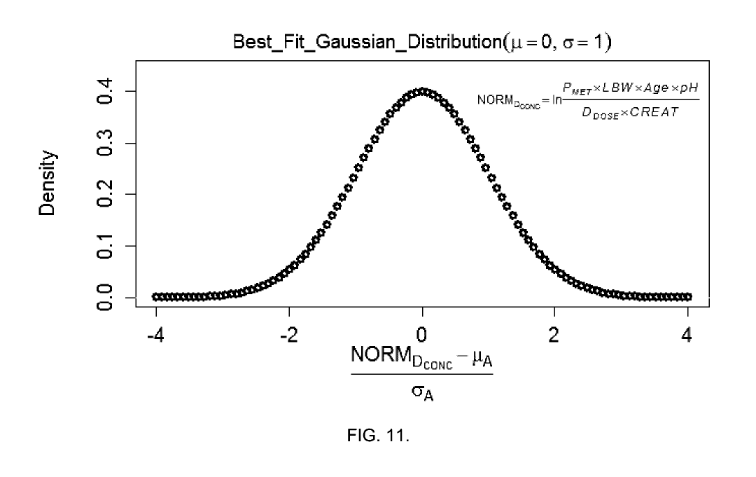

FIG. 12 shows an overlay of the least squares minimized best fit Gaussian distribution (circles) and the kernel density estimation plot (solid line) derived from the normalized, transformed, and standardized data from FIG. 1 (i.e., FIG. 10).

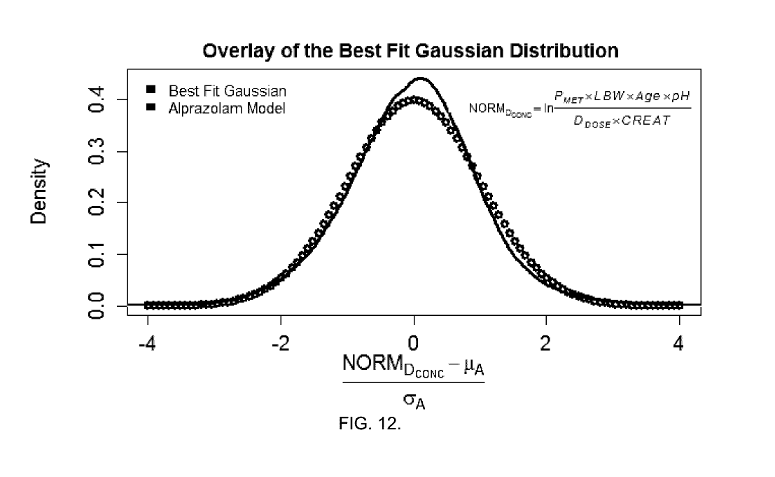

FIG. 13 shows a histogram of the hydrocodone drug concentrations observed from a body of collected urine test results used to generate the mathematically normalized and transformed standard curve for hydrocodone from Urine.

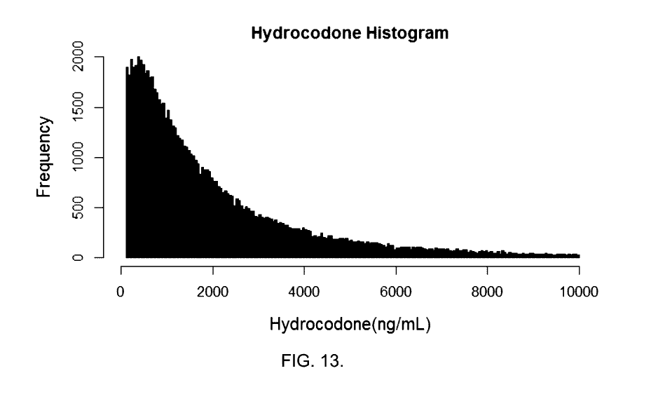

FIG. 14 shows the corresponding kernel density estimation plot derived from the data in FIG. 13.

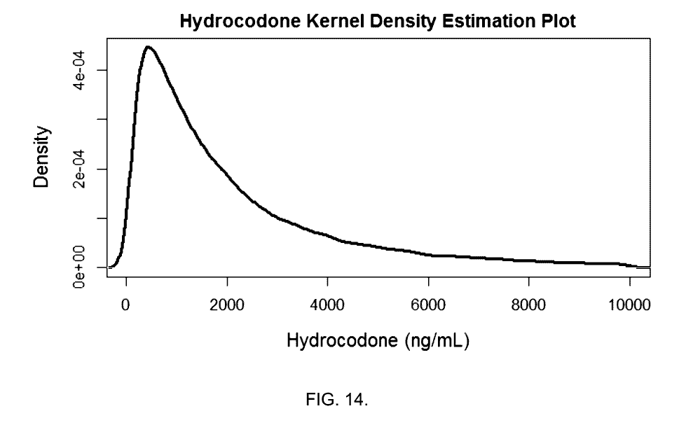

FIG. 15 shows the impact of mathematically normalizing, transforming, and standardizing the data presented in FIG. 13 using subject specific parameters or transformed variables arising from these parameters.

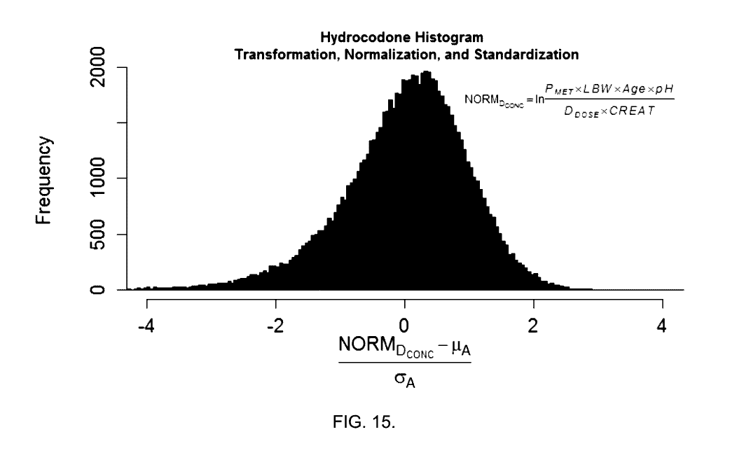

FIG. 16 shows the corresponding kernel density estimation plot derived from the mathematically normalized, transformed, and standardized data presented in FIG. 15.

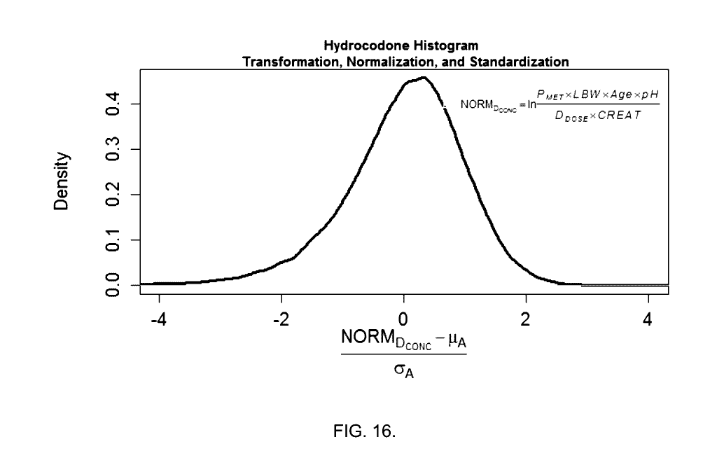

FIG. 17 shows a least squares minimized best fit Gaussian distribution derived from the normalized, transformed, and standardized data from FIG. 1 (i.e., FIG. 16).

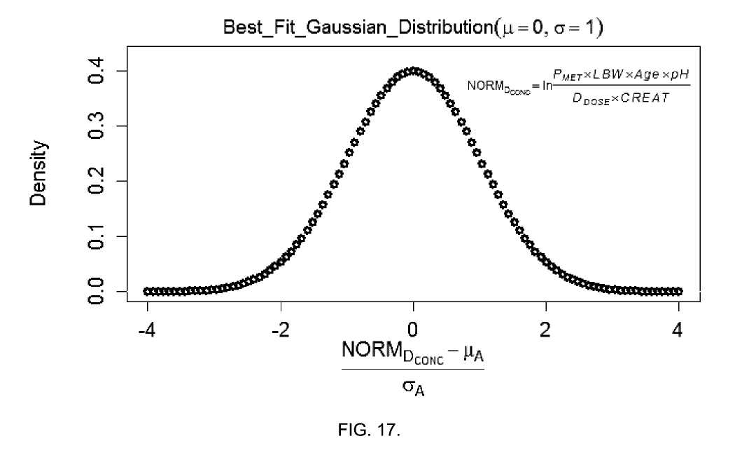

FIG. 18 shows an overlay of the least squares minimized best fit Gaussian distribution (circles) and the kernel density estimation plot (solid line) derived from the normalized, transformed, and standardized data from FIG. 1 (i.e., FIG. 16).

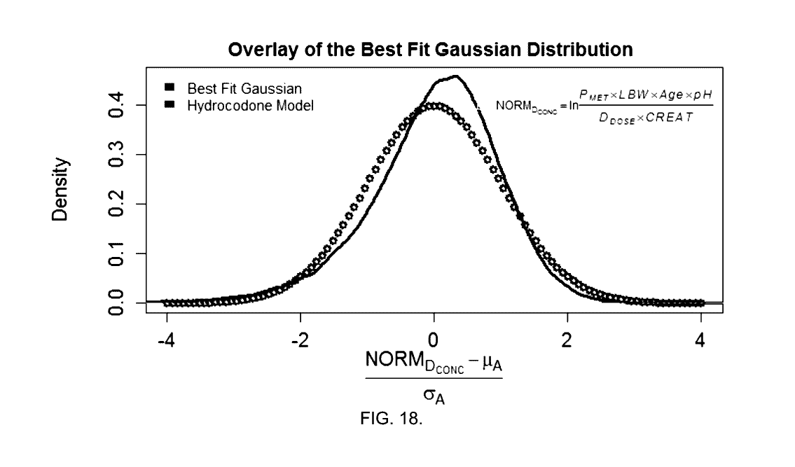

FIG. 19 shows a histogram of the oxycodone drug concentrations observed from a body of collected urine test results used to generate the mathematically normalized and transformed standard curve for oxycodone from Urine.

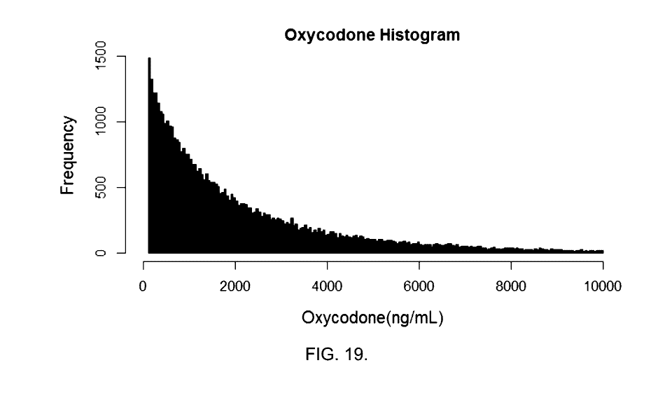

FIG. 20 shows the corresponding kernel density estimation plot derived from the data in FIG. 19.

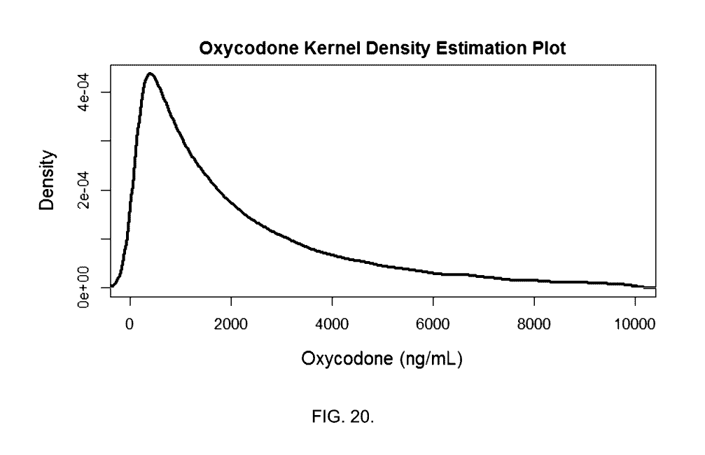

FIG. 21 shows the impact of mathematically normalizing, transforming, and standardizing the data presented in FIG. 19 using subject specific parameters or transformed variables arising from these parameters.

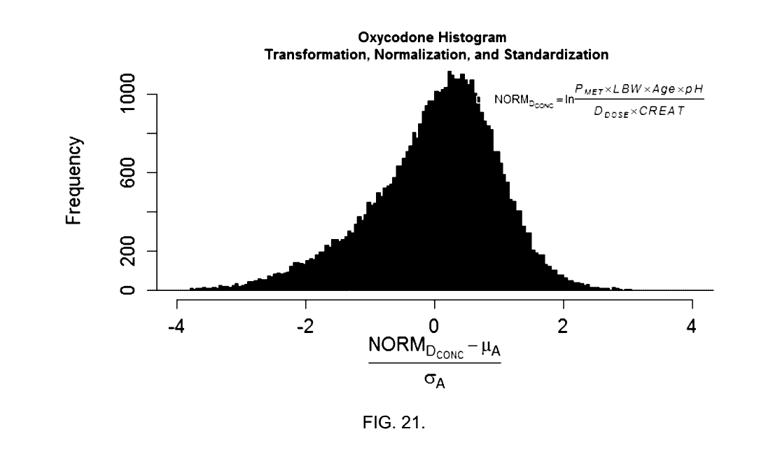

FIG. 22 shows the corresponding kernel density estimation plot derived from the mathematically normalized, transformed, and standardized data presented in FIG. 21.

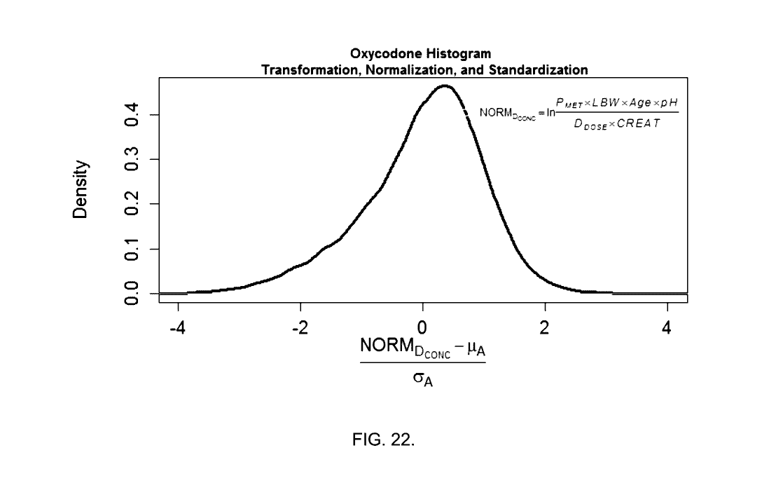

FIG. 23 shows a least squares minimized best fit Gaussian distribution derived from the normalized, transformed, and standardized data from FIG. 19 (i.e., FIG. 22).

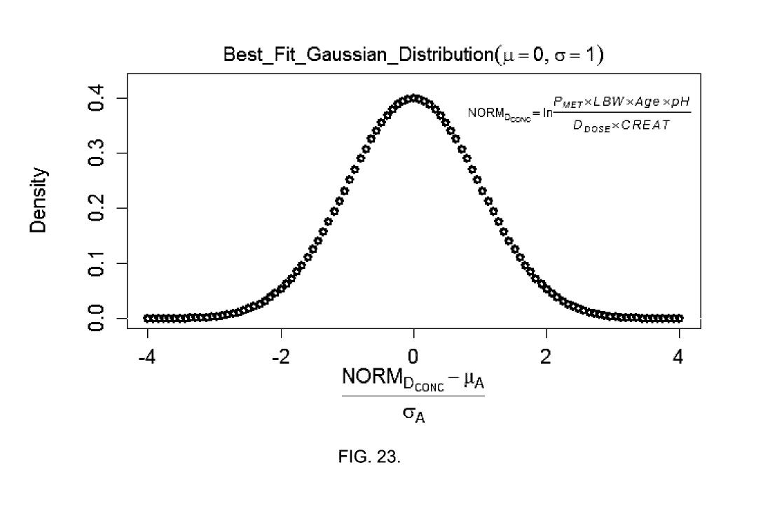

FIG. 24 shows an overlay of the least squares minimized best fit Gaussian distribution (circles) and the kernel density estimation plot (solid line) derived from the normalized, transformed, and standardized data from FIG. 19 (i.e., FIG. 22).

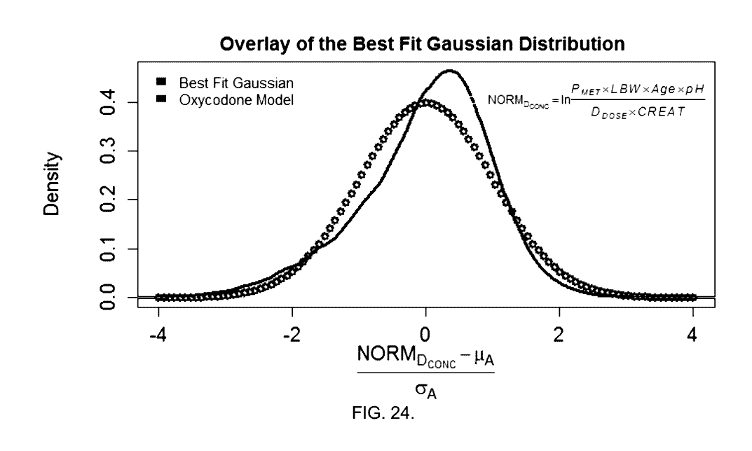

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the methods of the present disclosure.

As used herein, the singular form of a word includes the plural, and vice versa, unless the context clearly dictates otherwise. Thus, the references “a”, “an”, and “the” are generally inclusive of the plurals of the respective terms. For example, reference to “an embodiment” or “a method” includes a plurality of such “embodiments” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. The terms “comprising” or “including” are intended to include embodiments encompassed by the terms “consisting essentially of” and “consisting of.” Similarly, the term “consisting essentially of” is intended to include embodiments encompassed by the term “consisting of”.

### Therapeutic Regimens

In one embodiment, the present disclosure provides a method to assist in detecting non-compliance or potential non-compliance with a prescribed drug regimen (e.g., a prescribed regimen that includes buprenorphine therapy, or hydrocodone therapy, or oxycodone therapy) in a subject. The term “non-compliance” as used herein refers to any substantial deviation from a course of treatment that has been prescribed by a physician, nurse, nurse practitioner, physician's assistant, or other health care professional. A substantial deviation from a course of treatment may include any intentional or unintentional behavior by the subject that increases or decreases the amount, timing or frequency of drug ingested or otherwise administered (e.g., transdermal patch) compared to the prescribed therapy.

Non-limiting examples of substantial deviations from a course of treatment include: taking more of the drug than prescribed, taking less of the drug than prescribed, taking the drug more often than prescribed, taking the drug less often than prescribed, intentionally diverting at least a portion of the prescribed drug, unintentionally diverting at least a portion of the prescribed drug, etc. For example, a subject substantially deviates from a course of treatment by taking about 5% to about 1000% of the prescribed daily dose or prescribed drug regimen, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 105%, about 110%, about 115%, about 120%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% of the prescribed drug regimen.

A subject may also substantially deviate from a course of treatment by taking about 5% to about 1000% more or less than the prescribed dose, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% less than the prescribed dose. A subject may also substantially deviate from a course of treatment by, for example, taking the prescribed dose of a drug about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% more often or less often than specified in the course of treatment or prescribed in the drug regimen.

In some embodiments, a subject according to the present disclosure is prescribed a daily dose of a drug (e.g., a drug comprising, consisting essentially of, or consisting of buprenorphine or hydrocodone or oxycodone, etc.). The term “daily dose” or “prescribed daily dose” as used herein refers to any periodic administration of a drug to the subject over a given period of time, for example per hour, per day, per every other day, per week, per month, per year, etc. Preferably the daily dose or prescribed daily dose is the amount of the drug prescribed to a subject in any 24-hour period. While the drug may be administered according to any method known in the art including, for example, orally, intravenously, topically, transdermally, subcutaneously, sublingually, rectally, etc., for the purposes of this provisional application, the test results must be derived from urine samples. The prescribed daily dose of the drug may be approved by the Food & Drug Administration (“FDA”) for a given indication. In the alternative, a daily dose or a prescribed daily dose may be an unapproved or “off-label” use for a drug for which FDA has approved other indications. As a non-limiting example, FDA has approved buprenorphine sublingual films or tablets (SUBOXONE®) and buprenorphine hydrochloride (SUBUTEX®) for use in the treatment of opioid dependence in 2 mg and 8 mg tablets and in lowers doses for pain management in non-opioid-tolerant patients. Any use of buprenorphine sublingual films or tablets (SUBOXONE®) and buprenorphine hydrochloride (SUBUTEX®) other than to treat opioid dependence or pain management or at other than approved doses is an “off-label” use.

In various embodiments, methods according to the present disclosure involve the step of determining a prescribed dose of a drug (e.g., buprenorphine or hydrocodone or oxycodone, etc.). The term “determining a prescribed dose” as used herein refers to any method known to those in the art to ascertain, discover, deduce, or otherwise learn the dose of a particular drug that has been prescribed to the subject. Non-limiting examples include subject interview, consultation with the subject's medical history, consultation with another health care professional familiar with the subject, consultation with a medical record associated with the subject, etc.

In some embodiments, the drug is an opioid. The term “opioid” as used herein refers to any natural, endogenous, synthetic, or semi-synthetic compound that binds to opioid receptors. Non-limiting examples of opioids include: codeine, morphine, thebaine, oripavine, diacetylmorphine, dihydrocodeine, hydrocodone, hydromorphone, nicomorphone, oxycodone, oxymorphone, fentanyl, alphamethylfentanyl, alfentanil, sufentanil, remifentanil, carfentanyl, ohmefentanyl, pethidine, keobemidone, desmethylprodine, (“MPPP”), allylprodine, prodine, 4-phenyl-1-(2-phenylethyl)piperidin-4-yl acetate (“PEPAP”), propoxyphene, dextropropoxyphene, dextromoramide, bezitramide, piritramide, methadone, dipipanone, levomathadyl acetate (“LAAM”), difenoxin, diphenoxylate, loperamide, dezocine, pentazocine, phenazocine, buprenorphine, dihydroetorphine, etorphine, butorphanol, nalbuphine, levorphanol, levomethorphan, lefetamine, meptazinol, tilidine, tramadol, tapentadol, nalmefene, naloxone, naltrexone, methadone, derivatives thereof, metabolites thereof, prodrugs thereof, controlled-release formulations thereof, extended-release formulations thereof, sustained-release formulations thereof, and combinations of the foregoing.

In an embodiment, a method according to the present disclosure confirms a subject's non-adherence to a chronic buprenorphine therapy. For example, the term “chronic buprenorphine therapy” as used herein refers to any short-term, mid-term, or long-term treatment regimen comprising buprenorphine. As a non-limiting example, a subject suffering Opioid addiction may ingest a daily dose of buprenorphine to help wean them off of their addition. In one embodiment, a method according to the present disclosure confirms (e.g., to a health care professional) a subject's adherence or non-adherence to a chronic buprenorphine therapy. In some embodiments, the chronic buprenorphine therapy is a component of a therapeutic treatment regimen, such as addiction therapy.

In an embodiment, a method according to the present disclosure confirms a subject's non-adherence to a Chronic Opioid Therapy (COT). The term “chronic opioid therapy” as used herein refers to any short-term, mid-term, or long-term treatment regimen comprising an opioid pain drug. As a non-limiting example, a subject suffering chronic pain may ingest a daily dose of hydrocodone to relieve persistent pain resulting from trauma, chronic conditions, etc. In one embodiment, a method according to the present disclosure confirms (e.g., to a health care professional) a subject's adherence or non-adherence to a chronic hydrocodone therapy. In some embodiments, the chronic hydrocodone therapy is a component of a therapeutic treatment regimen, such as chronic opioid therapy (“COT”).

As another non-limiting example, a subject suffering chronic pain may ingest a daily dose of oxycodone to relieve persistent pain resulting from trauma, chronic conditions, etc. In one embodiment, a method according to the present disclosure confirms (e.g., to a health care professional) a subject's adherence or non-adherence to a chronic oxycodone therapy. In some embodiments, the chronic oxycodone therapy is a component of a therapeutic treatment regimen, such as chronic opioid therapy (“COT”).

Subjects on COT sometimes develop an addiction to the prescribed opioid. Studies have shown that a subject on COT is more likely to develop an addiction to a prescribed opioid when he or she has a history of aberrant drug-related behavior, or is at high risk of aberrant drug-related behavior. The term “aberrant drug-related behavior” as used herein refers to any behavioral, genetic, social, or other characteristic of the subject that tends to predispose the subject to development of an addiction for an opioid.

Non-limiting examples of such risk factors include a history of drug abuse, a history of opioid abuse, a history of non-opioid drug abuse, a history of alcohol abuse, a history of substance abuse, a history of prescription drug abuse, a low tolerance to pain, a high rate of opioid metabolism, a history of purposeful over-sedation, negative mood changes, intoxicated appearance, an increased frequency of appearing unkempt or impaired, a history of auto or other accidents, frequent early renewals of prescription medications, a history of or attempts to increasing dose without authorization, reports of lost or stolen medications, a history of contemporaneously obtaining prescriptions from more than one doctor, a history of altering the route of administering drugs, a history of using pain relief medications in response to stressful situations, insistence on certain medications, a history of contact with street drug culture, a history of alcohol abuse, a history of illicit drug abuse, a history of hoarding or stockpiling medications, a history of police arrest, instances of abuse or violence, a history of visiting health care professionals without an appointment, a history of consuming medications in excess of the prescribed dose, multiple drug allergies and/or intolerances, frequent office calls and visits, a genetic mutation that up-regulates or down-regulates production of drug metabolizing enzymes, a reduced-function CYP2D6 allele, and/or a non-functional CYP2D6 allele.

In an embodiment, a method according to the present disclosure confirms a subject's non-adherence to an opioid addiction treatment. The term “opioid addiction” as used herein refers to a neurobehavioral syndrome characterized by the repeated compulsive seeking or use of an opioid despite adverse social, psychological and/or physical consequences (Substance Abuse and Mental Health Services Administration, 2012). As a non-limiting example, a subject suffering from opioid dependence may be treated with pharmacotherapy with partial-agonist maintenance with buprenorphine or buprenorphine/naloxone; such subjects on pharmacotherapy are recommended for periodic monitoring by a health care professional for adherence to the regimen. Routine testing is also recommended for new subjects being considered for partial agonist maintenance to confirm buprenorphine is not already present in the subject's system. Routine testing is also recommended for subjects prescribed partial agonist maintenance to confirm buprenorphine and/or naloxone is/are not present in the subject's system as a result of chronic therapy.

In one embodiment, a method according to the present disclosure assists a health care professional in confirming a subject's adherence or non-adherence to an opioid dependence treatment regimen; e.g. buprenorphine and naloxone (e.g., Suboxone) treatment.

In an embodiment, the present disclosure assesses or determines a risk that a subject is misusing a prescribed drug (e.g., buprenorphine and/or naloxone), of particular importance in monitoring opioid addiction therapy. In some embodiments, the determined risk is communicated to a health care professional. For example, based on the comparison of the mathematically normalized and transformed datum to the same mathematically normalized and transformed standard distribution performed in embodiments of the present disclosure, a healthcare worker can intervene (e.g. via counseling, modifying the subject's regiment/dose, etc.) in the subject's misuse on the basis of the risk assessment.

### Sample Measurement

Methods according to the present disclosure may be used to determine the comparison of a mathematically normalized and transformed datum to a similarly normalized and transformed standard distribution of a wide variety of drugs in urine of a subject. When the fluid analyzed is urine, for example, methods according to the present disclosure may be used to determine the comparison of any drug that can be measured in a urine sample to a like standard distribution. In some embodiments, the drug comprises buprenorphine.

In an embodiment, the amount of a drug (e.g., buprenorphine) in a subject is determined by analyzing a fluid of the subject. The term “fluid” as used herein refers to urine and any liquid or pseudo-liquid obtained from the urinary track of the subject. Non-limiting examples include urine and the like. In an embodiment, the fluid is urine.

Determining the amount of a drug (e.g., buprenorphine) in urine of the subject may be accomplished by use of any method known to those skilled in the art. Non-limiting examples for determining the amount of a drug in fluid of a subject include fluorescence polarization immunoassay (“FPIA,” Abbott Diagnostics), mass spectrometry (MS), gas chromatography-mass spectrometry (GC-MS-MS), liquid chromatography-mass spectrometry (LC-MS-MS), liquid chromatography—exact mass mass spectrometry (LC/QTOF) and the like. In one embodiment, LC-MS-MS methods known to those skilled in the art are used to determine a raw level, amount, or concentration of a drug (e.g., buprenorphine) in urine of the subject. In one embodiment, a raw level, amount, or concentration of a drug in urine of a subject is measured and reported as a ratio, percent, or in relationship to the amount of fluid. The amount of fluid may be expressed as a unit volume, for example, in L, mL, μL, pL, ounce, etc. In one embodiment, the raw amount of a drug in Urine of a subject may be expressed as an absolute level or value, for example, in g, mg, μg, ng, pg, etc.

In an embodiment, the level, concentration, or amount of a drug (e.g., buprenorphine) determined in urine of a subject is normalized. The term “normalized” as used herein refers to a level, amount, or concentration of a drug that has been adjusted to correct for one or more parameters associated with the subject. Non-limiting examples of parameters include: sample fluid pH, sample fluid specific gravity, sample fluid creatinine concentration, sample fluid salt concentration, sample fluid osmolality, sample fluid uric acid concentration, subject height, subject weight, subject age, subject body mass index, subject gender, subject lean body mass, subject calculated blood volume, subject total body water volume, and subject body surface area, subject prescribed drug dosage. Parameters may be measured by any means known in the art. For example, sample fluid pH may be measured using a pH meter, litmus paper, test strips, etc. Uric acid is the end product in humans of purine metabolism and thus reflects the general metabolic profile of the patient. Uric acid values range from 3.4 to 7.2 mg/dL in men and 2.4 to 6.1 mg/dL in women with elevated uric acid levels resulting in the arthritic condition known as Gout.

In some embodiments, the normalized drug concentration is determined using parameters comprising, consisting essentially of, or consisting of sample pH, sample fluid creatinine concentration, subject height, subject weight, subject gender, subject body mass index, subject lean body weight, subject body surface area, subject prescribed drug dosage, and subject age. In other embodiments, the normalized drug concentration is determined from the primary metabolite concentration using parameters comprising, consisting essentially of, or consisting of subject height, subject weight, subject gender, subject body mass index, subject lean body weight, subject prescribed drug dosage, and sample fluid creatinine concentration. In yet other embodiments, the normalized drug concentration is determined from the primary metabolite concentration and the secondary metabolite concentration using parameters consisting of primary metabolite concentration, secondary metabolite concentration, subject height, subject weight, subject gender, subject body mass index, subject lean body weight, subject body surface area, subject prescribed drug dosage, and sample fluid creatinine concentration. The parent drug is also referred to as the primary metabolite.

In some embodiments, the normalized drug concentration is determined from the primary metabolite concentration comprising, consisting essentially of, or consisting of sample pH, subject weight, subject height, subject gender, subject age, sample creatinine concentration, and prescribed daily dose.

In some embodiments, the normalized drug concentration is determined from the primary metabolite concentration comprising, consisting essentially of, or consisting of subject height, subject weight, subject gender, prescribed daily dose, and sample creatinine concentration.

In some embodiments, the normalized drug concentration is determined from the primary metabolite concentration comprising, consisting essentially of, or consisting of prescribed daily dose and sample creatinine concentration.

In some embodiments, the normalized drug concentration is determined from the primary metabolite concentration comprising, consisting essentially of, or consisting of sample creatinine concentration.

In an embodiment, once the level, concentration, or amount of a drug determined in urine of a subject is normalized, it is then transformed. The term “transformed” as used herein refers to a mathematical operation on the level or concentration of a drug that has been adjusted to correct for one or more parameters associated with the subject (i.e., “normalized”). Transformation is a recognized mathematical operation that takes “data” from one “space” into another “space”. Examples of transformations include but are not limited to the first derivative of the adjusted data, the integral of the adjusted data over all concentration, applying polar coordinates to Cartesian data, taking the inverse of the adjusted data (i.e., 1/X), and taking the adjusted data from linear space to natural logarithm space. It is understood that a complete list of transformations is difficult if not impossible to place herein. Thus, any and all transformations of the adjusted (i.e., “normalized”) data are disclosed herein. The natural log transformation is of particular importance in methods of the current disclosure but is not the only transformation that will provide adequate standard distributions of the population of data to be used in these curves.

In an embodiment, the raw drug concentration measured in Urine of the subject is normalized as a function of subject height, subject weight, subject gender, subject age, subject lean body weight, subject prescribed drug dosage, sample fluid pH, and sample fluid creatinine concentration and then transformed through the natural logarithm. (hereafter “Equation 1”):

$${NORM}_{{D}_{CONC}} = ln ( \frac{{P}_{MET} * LBW * Age * pH}{{D}_{DOSE} * CREAT} ) \qquad ( 1 )$$

Where ln is the natural log, P_MET is the concentration of the primary metabolite also referred to as the parent drug in kg/L; LBW is the lean body weight of the subject in kg; Age is the subject age in years; pH is the sample fluid pH; D_DOSE is the subject prescribed drug dosage in kg; and CREAT is the sample fluid creatinine concentration in kg/L. The value is then transformed into its corresponding value on the standard normal (e.g., Gaussian) distribution using (hereafter “Equation 1-A):

$${NORM}_{STD ( A )} = \frac{( {NORM}_{{D}_{CONC}} - {μ}_{A} )}{{σ}_{A}} \qquad ( 1 \text{-} A )$$

where NORM_{STD(A)} is the standardized normal value and μ_{A} and σ_{A} are the mean and the standard deviation of the population used to construct the model described in Equation 1. The resulting mean and standard deviation of the standardized normal distribution, NORM_{STD(A)}, are “0” and “1” respectively.

In an embodiment, if the primary metabolite concentration is measured as zero or below the limit of detection of the method for a patient prescribed the drug, Equation 1 cannot be utilized and said patient will be deemed as potentially non-compliant. Alternatively, in the case where the primary metabolite concentration is less than the analytical method limit of quantitation (LOQ), a predetermined minimum value can be used to describe the data. As a non-limiting example, the predetermined minimum primary metabolite value and/or the predetermined minimum secondary metabolite value for use in Equation 1 can be 10 ng/mL or 1×10⁻⁸ kg/L. As another non-limiting example, the predetermined minimum primary metabolite value and/or the predetermined minimum secondary metabolite value for use in Equation 1 can be 10 ng/mL or 1×10⁻⁸ kg/L. As yet another non-limiting example, the predetermined minimum primary metabolite value and/or the predetermined minimum secondary metabolite value for use in Equation 1 can be 1 ng/mL or 1×10⁻⁹ kg/L. As a non-limiting example, the predetermined minimum primary metabolite value and/or the predetermined minimum secondary metabolite value for use in Equation 1 can be as low as the method of detection is capable of quantitating the value (e.g., Limit of Quantitation) which is dependent upon instrumentation and sample preparation as is well known by those skilled in the art. Finally, in the absence of any guidance from the literature or other analytical methods, the value for samples below the determined limit of quantitation can arbitrarily be assigned a value equal to 50% of the determined limit of quantitation, more preferably 40% of the determined limit of quantitation, and most preferably 30% of the determined limit of quantitation.

In an embodiment, if the primary metabolite concentration is measured as zero, the primary metabolite concentration is used in Equation 1 as a different value, such as, for example, a predetermined minimum primary metabolite value for use in Equation 1. Additionally or alternatively, if the secondary metabolite concentration is measured as zero, the secondary metabolite concentration is used in Equation 1 as a different value, such as, for example, a predetermined minimum secondary metabolite value for use in Equation 1. As a non-limiting example, the predetermined minimum primary metabolite value and/or the predetermined minimum secondary metabolite value for use in Equation 1 can be 5 ng/mL. As a non-limiting example, the predetermined minimum primary metabolite value and/or the predetermined minimum secondary metabolite value for use in Equation 1 can be 1 ng/mL. As a non-limiting example, the predetermined minimum primary metabolite value and/or the predetermined minimum secondary metabolite value for use in Equation 1 can be 0.1 ng/mL

In a related embodiment, for a subject prescribed buprenorphine, a normalized drug level is determined from a raw level of the primary metabolite or the secondary metabolite as a function of subject height, subject weight, subject gender, subject age, subject lean body weight, subject prescribed drug dosage, sample fluid pH, and sample fluid creatinine concentration, according to Equation 1. In a related embodiment, buprenorphine is the only opioid prescribed to the subject.

In a related embodiment, for a subject prescribed buprenorphine sublingual films or tablets (SUBOXONE®, Temgesic), a normalized drug level is determined from a raw level of the primary metabolite and the secondary metabolite as a function of subject height, subject weight, subject gender, subject age, subject lean body weight, subject prescribed drug dosage, sample fluid pH, and sample fluid creatinine concentration, according to Equation 1. In a related embodiment, sublingual films or tablets (SUBOXONE®, Temgesic) is the only opioid prescribed to the subject.

In a related embodiment, for a subject prescribed buprenorphine hydrochloride (SUBUTEX®), a normalized drug level is determined from a raw level of the primary metabolite and the secondary metabolite as a function of subject height, subject weight, subject gender, subject age, subject lean body weight, subject prescribed drug dosage, sample fluid pH, and sample fluid creatinine concentration, according to Equation 1. In a related embodiment, buprenorphine hydrochloride (SUBUTEX®) is the only opioid prescribed to the subject.

In a related embodiment, for a subject prescribed buprenorphine transdermal patch (Butrans®), a normalized drug level is determined from a raw level of the primary metabolite and the secondary metabolite as a function of subject height, subject weight, subject gender, subject age, subject lean body weight, subject prescribed drug dosage, sample fluid pH, and sample fluid creatinine concentration, according to Equation 1. In a related embodiment, buprenorphine transdermal patch (Butrans®) is the only opioid prescribed to the subject.

In an embodiment, the raw drug concentration measured in urine of the subject is normalized as a function of subject height, subject weight, subject lean body weight, subject prescribed drug dosage, sample fluid pH, and sample fluid creatinine concentration and transformed via the natural logarithm (hereafter “Equation 2”):

$${NORM}_{{D}_{CONC}} = ln ( \frac{{P}_{MET} * LBW * pH}{{D}_{DOSE} * CREAT} ) \qquad ( 2 )$$

Where ln is the natural log, P_MET is the concentration of the primary metabolite also referred to as the parent drug in kg/L; LBW is the lean body weight of the subject in kg; pH is the sample fluid pH; D_DOSE is the subject prescribed drug dosage in kg; and CREAT is the sample fluid creatinine concentration in kg/L. The value is then transformed into its corresponding value on the standard normal distribution using (hereafter “Equation 2-A):

$${NORM}_{STD ( B )} = \frac{( {NORM}_{{D}_{CONC}} - {μ}_{B} )}{{σ}_{B}} \qquad ( 2 \text{-} A )$$

where NORM_{STD(B)} is the standardized normal value and μ_{B} and σ_{B} are the mean and the standard deviation of the population used to construct the model described in Equation 2. The resulting mean and standard deviation of the standardized normal distribution, NORM_{STD(B)}, are “0” and “1” respectively.

In an embodiment, if the primary metabolite concentration is measured as zero or below the limit of detection of the method for a patient prescribed the drug, Equation 2 cannot be utilized and said patient will be deemed as potentially non-compliant. Alternatively, in the case where the primary metabolite concentration is less than the analytical method limit of quantitation (LOQ), a predetermined minimum value can be used to describe the data. As a non-limiting example, the predetermined minimum primary metabolite value and/or the predetermined minimum secondary metabolite value for use in Equation 2 can be 10 ng/mL or 1×10⁻⁸ kg/L. As another non-limiting example, the predetermined minimum primary metabolite value and/or the predetermined minimum secondary metabolite value for use in Equation 2 can be 10 ng/mL or 1×10⁻⁸ kg/L. As yet another non-limiting example, the predetermined minimum primary metabolite value and/or the predetermined minimum secondary metabolite value for use in Equation 2 can be 1 ng/mL or 1×10⁻⁹ kg/L. As a non-limiting example, the predetermined minimum primary metabolite value and/or the predetermined minimum secondary metabolite value for use in Equation 2 can be as low as the method of detection is capable of quantitating the value (e.g., Limit of Quantitation) which is dependent upon instrumentation and sample preparation as is well known by those skilled in the art. Finally, in the absence of any guidance from the literature or other analytical methods, the value for samples below the determined limit of quantitation can arbitrarily be assigned a value equal to 50% of the determined limit of quantitation, more preferably 40% of the determined limit of quantitation, and most preferably 30% of the determined limit of quantitation.

In an embodiment, if the primary metabolite concentration is measured as zero, the primary metabolite concentration is used in Equation 2 as a different value, such as, for example, a predetermined minimum primary metabolite value for use in Equation 2. Additionally or alternatively, if the secondary metabolite concentration is measured as zero, the secondary metabolite concentration is used in Equation 2 as a different value, such as, for example, a predetermined minimum secondary metabolite value for use in Equation 2. As a non-limiting example, the predetermined minimum primary metabolite value and/or the predetermined minimum secondary metabolite value for use in Equation 2 can be 5 ng/mL. As a non-limiting example, the predetermined minimum primary metabolite value and/or the predetermined minimum secondary metabolite value for use in Equation 2 can be 1 ng/mL. As a non-limiting example, the predetermined minimum primary metabolite value and/or the predetermined minimum secondary metabolite value for use in Equation 2 can be 0.1 ng/mL

In a related embodiment, for a subject prescribed buprenorphine, a normalized drug level is determined from a raw level of the primary metabolite or the secondary metabolite as a function of subject height, subject weight, subject gender, subject lean body weight, subject prescribed drug dosage, sample fluid pH, and sample fluid creatinine concentration, according to Equation 2. In a related embodiment, buprenorphine is the only opioid prescribed to the subject.

In a related embodiment, for a subject prescribed buprenorphine sublingual films or tablets (SUBOXONE®, Temgesic), a normalized drug level is determined from a raw level of the primary metabolite and the secondary metabolite as a function of subject height, subject weight, subject gender, subject lean body weight, subject prescribed drug dosage, sample fluid pH, and sample fluid creatinine concentration, according to Equation 2. In a related embodiment, sublingual films or tablets (SUBOXONE®, Temgesic) is the only opioid prescribed to the subject.

In a related embodiment, for a subject prescribed buprenorphine hydrochloride (SUBUTEX®), a normalized drug level is determined from a raw level of the primary metabolite and the secondary metabolite as a function of subject height, subject weight, subject gender, subject lean body weight, subject prescribed drug dosage, sample fluid pH, and sample fluid creatinine concentration, according to Equation 2. In a related embodiment, buprenorphine hydrochloride (SUBUTEX®) is the only opioid prescribed to the subject.

In a related embodiment, for a subject prescribed buprenorphine hydrochloride (Butrans®), a normalized drug level is determined from a raw level of the primary metabolite and the secondary metabolite as a function of subject height, subject weight, subject gender, subject lean body weight, subject prescribed drug dosage, sample fluid pH, and sample fluid creatinine concentration, according to Equation 2. In a related embodiment, buprenorphine hydrochloride (Butrans®) is the only opioid prescribed to the subject.

In an embodiment, the raw drug concentration measured in Urine of the subject is normalized as a function of subject height, subject weight, subject gender, subject lean body weight, subject prescribed drug dosage, and sample fluid creatinine concentration and transformed through the natural logarithm. (hereafter “Equation 3”):

$${NORM}_{{D}_{CONC}} = ln ( \frac{{P}_{MET} * LBW}{{D}_{DOSE} * CREAT} ) \qquad ( 3 )$$

Where ln is the natural log, P_MET is the concentration of the primary metabolite also referred to as the parent drug in kg/L; LBW is the lean body weight of the subject in kg; D_DOSE is the subject prescribed drug dosage in kg; and CREAT is the sample fluid creatinine concentration in kg/L. The value is then transformed into its corresponding value on the standard normal distribution using (hereafter “Equation 3-A):

$${NORM}_{STD ( C )} = \frac{( {NORM}_{{D}_{CONC}} - {μ}_{C} )}{{σ}_{C}} \qquad ( 3 \text{-} A )$$

where NORM_{STD(c)} is the standardized normal value and μ_{c} and σ_{c} are the mean and the standard deviation of the population used to construct the model described in Equation 3. The resulting mean and standard deviation of the standardized normal distribution, NORM_{STD(c)}, are “0” and “1” respectively.

In an embodiment, if the primary metabolite concentration is measured as zero or below the limit of detection of the method for a patient prescribed the drug, Equation 3 cannot be utilized and said patient will be deemed as potentially non-compliant. Alternatively, in the case where the primary metabolite concentration is less than the analytical method limit of quantitation (LOQ), a predetermined minimum value can be used to describe the data. As a non-limiting example, the predetermined minimum primary metabolite value and/or the predetermined minimum secondary metabolite value for use in Equation 3 can be 10 ng/mL or 1×10⁻⁸ kg/L. As another non-limiting example, the predetermined minimum primary metabolite value and/or the predetermined minimum secondary metabolite value for use in Equation 3 can be 10 ng/mL or 1×10⁻⁸ kg/L. As yet another non-limiting example, the predetermined minimum primary metabolite value and/or the predetermined minimum secondary metabolite value for use in Equation 3 can be 1 ng/mL or 1×10⁻⁹ kg/L. As a non-limiting example, the predetermined minimum primary metabolite value and/or the predetermined minimum secondary metabolite value for use in Equation 3 can be as low as the method of detection is capable of quantitating the value (e.g., Limit of Quantitation) which is dependent upon instrumentation and sample preparation as is well known by those skilled in the art. Finally, in the absence of any guidance from the literature or other analytical methods, the value for samples below the determined limit of quantitation can arbitrarily be assigned a value equal to 50% of the determined limit of quantitation, more preferably 40% of the determined limit of quantitation, and most preferably 30% of the determined limit of quantitation.

In an embodiment, if the primary metabolite concentration is measured as zero, the primary metabolite concentration is used in Equation 3 as a different value, such as, for example, a predetermined minimum primary metabolite value for use in Equation 3. Additionally or alternatively, if the secondary metabolite concentration is measured as zero, the secondary metabolite concentration is used in Equation 3 as a different value, such as, for example, a predetermined minimum secondary metabolite value for use in Equation 3. As a non-limiting example, the predetermined minimum primary metabolite value and/or the predetermined minimum secondary metabolite value for use in Equation 3 can be 5 ng/mL. As a non-limiting example, the predetermined minimum primary metabolite value and/or the predetermined minimum secondary metabolite value for use in Equation 3 can be 1 ng/mL. As a non-limiting example, the predetermined minimum primary metabolite value and/or the predetermined minimum secondary metabolite value for use in Equation 3 can be 0.1 ng/mL

In a related embodiment, for a subject prescribed buprenorphine, a normalized drug level is determined from a raw level of the primary metabolite or the secondary metabolite as a function of subject height, subject weight, subject gender, subject lean body weight, subject prescribed drug dosage, and sample fluid creatinine concentration, according to Equation 2. In a related embodiment, buprenorphine is the only opioid prescribed to the subject.

In a related embodiment, for a subject prescribed buprenorphine sublingual films or tablets (SUBOXONE®, Temgesic), a normalized drug level is determined from a raw level of the primary metabolite and the secondary metabolite as a function of subject height, subject weight, subject gender, subject lean body weight, subject prescribed drug dosage, and sample fluid creatinine concentration, according to Equation 3. In a related embodiment, sublingual films or tablets (SUBOXONE®, Temgesic) is the only opioid prescribed to the subject.

In a related embodiment, for a subject prescribed buprenorphine hydrochloride (SUBUTEX®), a normalized drug level is determined from a raw level of the primary metabolite and the secondary metabolite as a function of subject height, subject weight, subject gender, subject lean body weight, subject prescribed drug dosage, and sample fluid creatinine concentration, according to Equation 3. In a related embodiment, buprenorphine hydrochloride (SUBUTEX®) is the only opioid prescribed to the subject.

In a related embodiment, for a subject prescribed buprenorphine hydrochloride (Butrans®), a normalized drug level is determined from a raw level of the primary metabolite and the secondary metabolite as a function of subject height, subject weight, subject gender, subject lean body weight, subject prescribed drug dosage, and sample fluid creatinine concentration, according to Equation 3. In a related embodiment, buprenorphine hydrochloride (Butrans®) is the only opioid prescribed to the subject.

In an embodiment, the raw drug concentration measured in urine of the subject is normalized as a function of subject prescribed drug dosage and sample fluid creatinine concentration and transformed through the natural logarithm. (hereafter “Equation 4”):

$${NORM}_{{D}_{CONC}} = ln ( \frac{{P}_{MET}}{{D}_{DOSE} * CREAT} ) \qquad ( 4 )$$

Where ln is the natural log, P_MET is the concentration of the primary metabolite also referred to as the parent drug in kg/L; D_DOSE is the subject prescribed drug dosage in kg; and CREAT is the sample fluid creatinine concentration in kg/L. The value is then transformed into its corresponding value on the standard normal distribution using (hereafter “Equation 4-A):

$${NORM}_{STD ( D )} = \frac{( {NORM}_{{D}_{CONC}} - {μ}_{D} )}{{σ}_{D}} \qquad ( 4 \text{-} A )$$

where NORM_{STD(D)} is the standardized normal value and μ_{D} and σ_{D} are the mean and the standard deviation of the population used to construct the model described in Equation 4. The resulting mean and standard deviation of the standardized normal distribution, NORM_{STD(D)}, are “0” and “1” respectively.

In an embodiment, if the primary metabolite concentration is measured as zero or below the limit of detection of the method for a patient prescribed the drug, Equation 4 cannot be utilized and said patient will be deemed as potentially non-compliant. Alternatively, in the case where the primary metabolite concentration is less than the analytical method limit of quantitation (LOQ), a predetermined minimum value can be used to describe the data. As a non-limiting example, the predetermined minimum primary metabolite value and/or the predetermined minimum secondary metabolite value for use in Equation 4 can be 10 ng/mL or 1×10⁻⁸ kg/L. As another non-limiting example, the predetermined minimum primary metabolite value and/or the predetermined minimum secondary metabolite value for use in Equation 4 can be 10 ng/mL or 1×10⁻⁸ kg/L. As yet another non-limiting example, the predetermined minimum primary metabolite value and/or the predetermined minimum secondary metabolite value for use in Equation 4 can be 1 ng/mL or 1×10⁻⁹ kg/L. As a non-limiting example, the predetermined minimum primary metabolite value and/or the predetermined minimum secondary metabolite value for use in Equation 4 can be as low as the method of detection is capable of quantitating the value (e.g., Limit of Quantitation) which is dependent upon instrumentation and sample preparation as is well known by those skilled in the art. Finally, in the absence of any guidance from the literature or other analytical methods, the value for samples below the determined limit of quantitation can arbitrarily be assigned a value equal to 50% of the determined limit of quantitation, more preferably 40% of the determined limit of quantitation, and most preferably 30% of the determined limit of quantitation.

In an embodiment, if the primary metabolite concentration is measured as zero, the primary metabolite concentration is used in Equation 4 as a different value, such as, for example, a predetermined minimum primary metabolite value for use in Equation 4. Additionally or alternatively, if the secondary metabolite concentration is measured as zero, the secondary metabolite concentration is used in Equation 4 as a different value, such as, for example, a predetermined minimum secondary metabolite value for use in Equation 4. As a non-limiting example, the predetermined minimum primary metabolite value and/or the predetermined minimum secondary metabolite value for use in Equation 4 can be 5 ng/mL. As a non-limiting example, the predetermined minimum primary metabolite value and/or the predetermined minimum secondary metabolite value for use in Equation 4 can be 1 ng/mL. As a non-limiting example, the predetermined minimum primary metabolite value and/or the predetermined minimum secondary metabolite value for use in Equation 4 can be 0.1 ng/mL

In a related embodiment, for a subject prescribed buprenorphine, a normalized drug level is determined from a raw level of the primary metabolite or the secondary metabolite as a function of subject prescribed drug dosage and sample fluid creatinine concentration, according to Equation 4. In a related embodiment, buprenorphine is the only opioid prescribed to the subject.

In a related embodiment, for a subject prescribed buprenorphine sublingual films or tablets (SUBOXONE®, Temgesic), a normalized drug level is determined from a raw level of the primary metabolite and the secondary metabolite as a function of subject height, subject weight, subject lean body weight, subject prescribed drug dosage, and sample fluid creatinine concentration, according to Equation 4. In a related embodiment, sublingual films or tablets (SUBOXONE®, Temgesic) is the only opioid prescribed to the subject.

In a related embodiment, for a subject prescribed buprenorphine hydrochloride (SUBUTEX®), a normalized drug level is determined from a raw level of the primary metabolite and the secondary metabolite as a function of subject prescribed drug dosage and sample fluid creatinine concentration, according to Equation 4. In a related embodiment, buprenorphine hydrochloride (SUBUTEX®) is the only opioid prescribed to the subject.

In a related embodiment, for a subject prescribed buprenorphine hydrochloride (Butrans®), a normalized drug level is determined from a raw level of the primary metabolite and the secondary metabolite as a function of subject prescribed drug dosage and sample fluid creatinine concentration, according to Equation 4. In a related embodiment, buprenorphine hydrochloride (Butrans®) is the only opioid prescribed to the subject.

In an embodiment, the raw drug concentration measured in Urine of the subject is normalized as a function of only the sample fluid creatinine concentration and transformed through the natural logarithm. (hereafter “Equation 5”)

$${NORM}_{{D}_{CONC}} = ln ( \frac{{P}_{MET}}{CREAT} ) \qquad ( 5 )$$

Where ln is the natural log, P_MET is the concentration of the primary metabolite also referred to as the parent drug in kg/L and CREAT is the sample fluid creatinine concentration in kg/L. It is noted that this equation reflects the simple normalization and transformation (see 0007) that is often not Gaussian (i.e., skewed) and in use is not adjusted for offset from 0 (i.e., ADJ_E). clearly, the use of creatinine alone is not sufficient to model these data. The value is then transformed into its corresponding value on the standard normal distribution using (hereafter “Equation 5-A):

$${NORM}_{STD ( E )} = \frac{( {NORM}_{{D}_{CONC}} - {μ}_{E} )}{{σ}_{E}} \qquad ( 5 \text{-} A )$$

where NORM_{STD(E)} is the standardized normal value and μ_{E} and σ_{E} are the mean and the standard deviation of the population used to construct the model described in Equation 5. The resulting mean and standard deviation of the standardized normal distribution, NORM_{STD(E)}, are “0” and “1” respectively.

In an embodiment, if the primary metabolite concentration is measured as zero or below the limit of detection of the method for a patient prescribed the drug, Equation 5 cannot be utilized and said patient will be deemed as potentially non-compliant. Alternatively, in the case where the primary metabolite concentration is less than the analytical method limit of quantitation (LOQ), a predetermined minimum value can be used to describe the data. As a non-limiting example, the predetermined minimum primary metabolite value and/or the predetermined minimum secondary metabolite value for use in Equation 5 can be 10 ng/mL or 1×10⁻⁸ kg/L. As another non-limiting example, the predetermined minimum primary metabolite value and/or the predetermined minimum secondary metabolite value for use in Equation 5 can be 10 ng/mL or 1×10⁻⁸ kg/L. As yet another non-limiting example, the predetermined minimum primary metabolite value and/or the predetermined minimum secondary metabolite value for use in Equation 5 can be 1 ng/mL or 1×10⁻⁹ kg/L. As a non-limiting example, the predetermined minimum primary metabolite value and/or the predetermined minimum secondary metabolite value for use in Equation 5 can be as low as the method of detection is capable of quantitating the value (e.g., Limit of Quantitation) which is dependent upon instrumentation and sample preparation as is well known by those skilled in the art. Finally, in the absence of any guidance from the literature or other analytical methods, the value for samples below the determined limit of quantitation can arbitrarily be assigned a value equal to 50% of the determined limit of quantitation, more preferably 40% of the determined limit of quantitation, and most preferably 30% of the determined limit of quantitation.

In an embodiment, if the primary metabolite concentration is measured as zero, the primary metabolite concentration is used in Equation 5 as a different value, such as, for example, a predetermined minimum primary metabolite value for use in Equation 5. Additionally or alternatively, if the secondary metabolite concentration is measured as zero, the secondary metabolite concentration is used in Equation 5 as a different value, such as, for example, a predetermined minimum secondary metabolite value for use in Equation 5. As a non-limiting example, the predetermined minimum primary metabolite value and/or the predetermined minimum secondary metabolite value for use in Equation 5 can be 5 ng/mL. As a non-limiting example, the predetermined minimum primary metabolite value and/or the predetermined minimum secondary metabolite value for use in Equation 5 can be 1 ng/mL. As a non-limiting example, the predetermined minimum primary metabolite value and/or the predetermined minimum secondary metabolite value for use in Equation 5 can be 0.1 ng/mL

In a related embodiment, for a subject prescribed buprenorphine, a normalized drug level is determined from a raw level of the primary metabolite or the secondary metabolite as a function of subject prescribed drug dosage and sample fluid creatinine concentration, according to Equation 5. In a related embodiment, buprenorphine is the only opioid prescribed to the subject.

In a related embodiment, for a subject prescribed buprenorphine sublingual films or tablets (SUBOXONE®, Temgesic), a normalized drug level is determined from a raw level of the primary metabolite and the secondary metabolite as a function of subject height, subject weight, subject lean body weight, subject prescribed drug dosage, and sample fluid creatinine concentration, according to Equation 5. In a related embodiment, sublingual films or tablets (SUBOXONE®, Temgesic) is the only opioid prescribed to the subject.

In a related embodiment, for a subject prescribed buprenorphine hydrochloride (SUBUTEX®), a normalized drug level is determined from a raw level of the primary metabolite and the secondary metabolite as a function of subject prescribed drug dosage and sample fluid creatinine concentration, according to Equation 5. In a related embodiment, buprenorphine hydrochloride (SUBUTEX®) is the only opioid prescribed to the subject.

In a related embodiment, for a subject prescribed buprenorphine hydrochloride (Butrans®), a normalized drug level is determined from a raw level of the primary metabolite and the secondary metabolite as a function of subject prescribed drug dosage and sample fluid creatinine concentration, according to Equation 5. In a related embodiment, buprenorphine hydrochloride (Butrans®) is the only opioid prescribed to the subject.

In an embodiment, the concentration or level of drug in Urine of the subject is a steady state concentration or level. The term “steady state” as used herein refers to an equilibrium level or concentration of a drug obtained at the end of a certain number of administrations (e.g. 1 to about 5). Steady state is achieved when the concentration or level of the drug will remain substantially constant if the dose and the frequency of administrations remain substantially constant.

The parameters considered in the normalization for Equation 1, Equation 2, Equation 3, and Equation 4 include subject height, subject weight, subject gender, subject body mass index, subject lean body weight, subject prescribed drug dosage, sample fluid pH, and sample fluid creatinine concentration. All of these parameters were utilized in some modified or direct form in these mathematical normalized and transformed data points. Other than the patient creatinine levels, no other patient specific data were used in the normalization and transformation in Equation 5.

The lean body weight (LBW)—measured in kilograms—parameter accounts for the sum of everything in the human body with the exception of fat including but not limited to bones, muscles, and organs. The LBW is calculated using the James Formula (Absalom et al., 2009):

$$LBW = fact\_a * weight - fact\_b * {( \frac{weight}{100 * height} )}^{2} \qquad ( 6 )$$

Where fact_a equals 1.1 for Men and 1.07 for Women and fact_b equals 128 for Men and 148 for women. Weight is the subject weight measured in kg and height is the subject height in m.

In an embodiment, the normalized drug level obtained from Equation 1, Equation 2, Equation 3, Equation 4, and Equation 5 can be used in subsequent steps of the method, if any.

In an embodiment, Equation 1 is the most robust and preferred model used to determine whether the patients fall within the population of patients normally distributed around the standardized population mean.

In an embodiment, the distribution of transformed drug concentration data normalized using sample fluid creatinine concentration and using Equation 1, or Equation 2, or Equation 3, or Equation 4, or Equation 5, resembles a Gaussian distribution (a normally distributed symmetric bell curved function). In this population distribution, the distribution is standardized with the mean of the resulting population therefore being set to zero. The fitted population distribution therefore is predicted from statistics to exhibit 68% of the data within +/−1 standard deviation, 95% of the data within +/−2 standard deviations and the other 5% greater than +/−2 standard deviations. In order to access patient compliance it can be said that 95% of the time, compliant patients can be expected to fall within 95% of the data hence within +/−2 standard deviations of the population mean. Based on the design of these models any patient within +/−2 standard deviations of the population mean is likely to be complaint with their drug dosage regimen and the closer they are to the population mean, the more closely they resemble the patients whose parameters (raw drug concentration measured in Urine, subject height, subject weight, subject gender, subject lean body weight, subject prescribed drug dosage, sample fluid pH, and sample fluid creatinine concentration) resemble the mean of the population used to design the model. However, “compliant” is not a quantitative term in this respect and any patient that demonstrates data from Urine analysis which when mathematically normalized and transformed using sample fluid creatinine concentration and using Equation 1, or Equation 2, Equation 3, or Equation 4 falls within +/−2 standard deviations of the mathematically transformed and normalized standard distribution is likely “compliant”.

Subjects with mathematically normalized and transformed drug concentrations which fall outside +/−2 standard deviations of the corresponding mathematically normalized and transformed drug distribution may or may not be “compliant” in their adherence to their prescribed drug regimen. For example, for those subjects falling outside of −2 standard deviations from the mean of the standard distribution, it may be that they are ultra-rapid metabolizers and have cleared the drug from their blood volume (a CYP2D6 genetic issue), that they are not adherent; e.g. they are taking their drug less frequently than prescribed for any number of reasons such as expense, improved efficacy (less dose required), or in the worst case, they may be diverting their drug to a different use (e.g., for someone else, or for resale). On the other side, if their normalized and transformed drug concentration falls beyond +2 standard deviations from the mean of the standard distribution, it is possible that they are compliant but have very low metabolic rates (a different type of CYP2D6 genetic issue) leading to a build-up of drug in their blood and hence elevated drug concentrations in urine. Other reasons for high normalized and transformed drug concentrations could well result from noncompliance including taking larger amounts of drug than prescribed. In any event, the results of the comparison to the standard distribution will assist the health care provider with identifying adherence issues and resolving those issues to the benefit of the patient.

In a related embodiment, one or a plurality of subjects are assigned to a population. As used herein a “plurality of subjects” refers to two or more subjects, for example about 2 subjects, about 3 subjects, about 4 subjects, about 5 subjects, about 6 subjects, about 7 subjects, about 8 subjects, about 9 subjects, about 10 subjects, about 15 subjects, about 20 subjects, about 25 subjects, about 30 subjects, about 35 subjects, about 40 subjects, about 45 subjects, about 50 subjects, about 55 subjects, about 60 subjects, about 65 subjects, about 70 subjects, about 75 subjects, about 80 subjects, about 85 subjects, about 90 subjects, about 95 subjects, about 100 subjects, about 110 subjects, about 120 subjects, about 130 subjects, about 140 subjects, about 150 subjects, about 160 subjects, about 170 subjects, about 180 subjects, about 190 subjects, about 200 subjects, about 225 subjects, about 250 subjects, about 275 subjects, about 300 subjects, about 325 subjects, about 350 subjects, about 375 subjects, about 400 subjects, about 425 subjects, about 450 subjects, about 475 subjects, about 500 subjects, about 525 subjects, about 550 subjects, about 575 subjects, about 600 subjects, about 625 subjects, about 650 subjects, about 675 subjects, about 700 subjects, about 725 subjects, about 750 subjects, about 775 subjects, about 800 subjects, about 825 subjects, about 850 subjects, about 875 subjects, about 900 subjects, about 925 subjects, about 950 subjects, about 975 subjects, about 1000 subjects, about 1250 subjects, about 1500 subjects, about 1750 subjects, about 2000 subjects, about 2250 subjects, about 2500 subjects, about 2750 subjects, about 3000 subjects, about 3500 subjects, about 4000 subjects, about 4500 subjects, about 5000 subjects, about 5500 subjects, about 6000 subjects, about 6500 subjects, about 7000 subjects, about 7500 subjects, about 8000 subjects, about 8500 subjects, about 9000 subjects, about 9500 subjects, or about 10000 subjects. As used herein with respect to a population, the term “subject” is synonymous with the term “member” and refers to an individual that has been assigned to the population. In one embodiment, subpopulations may be established for a plurality of daily doses of a drug.

In an embodiment, a plurality of subjects of a population are each prescribed the same daily dose of a drug. In another embodiment, a plurality of subjects assigned to one subpopulation are each prescribed a first daily dose of a drug while a plurality of subjects assigned to a second, different subpopulation are each prescribed a second, different daily dose of a drug. In an embodiment, a plurality of subjects assigned to a population or subpopulation are each prescribed a daily dose of a drug for a time sufficient to achieve steady state. The term “time sufficient to achieve steady state” refers to the amount of time required, given the pharmacokinetics of the particular drug and the dose administered to the subject, to establish a substantially constant concentration or level of the drug assuming the dose and the frequency of administrations remain substantially constant. The time sufficient to achieve steady state may be determined from literature or other information corresponding to the drug. For example, labels or package inserts for FDA approved drugs often include information regarding typical times sufficient to achieve steady state plasma concentrations from initial dosing. Other non-limiting means to determine the time sufficient to achieve steady state include experiment, laboratory studies, analogy to similar drugs with similar absorption and excretion characteristics, etc.

Assignment of subjects to a population or subpopulation may be accomplished by any method known to those skilled in the art. For example, subjects may be assigned randomly to one of a plurality of subpopulations. In an embodiment, subjects are screened for one or more parameters before or after being assigned to a population. For example, subjects featuring one or more parameters that may tend to affect fluid levels of a drug may be excluded from a population, may not be assigned to a population, may be assigned to one of a plurality of subpopulations, or may be removed from a population or subpopulation during or after a data collection phase of a study. Subjects may be excluded from a population based on the presence or absence of one or more exclusion criteria such as high opioid metabolism, low opioid metabolism, lab abnormalities, impaired kidney or liver function, use of drugs with overlapping metabolites on the same day, excessive body weight or minimal body weight, or an inconsistent schedule of medication administration, as non-limiting examples.

The method may be used in combination with any other method known to those skilled in the art for detecting a subject's potential non-compliance with a prescribed treatment protocol based on the normalized variations of the population used to create these models. Non-limiting examples of such methods include: interviews with the subject, Oral fluid testing for the presence or absence of detectable levels of a drug, observation of the subject's behavior, appreciating reports of diversion of the subject's prescribed drug to others, etc.

In an embodiment, a method according to the present disclosure is used to reduce risk of drug misuse in a subject. In another embodiment, a method according to the present disclosure is used to confirm a subject's non-adherence to a chronic opioid therapy (COT) regimen. In yet another embodiment, a method according to the present disclosure provides a probability that a subject is non-compliant with a prescribed drug regimen. In an embodiment, a data point from the Urine testing of a subject is mathematically normalized and transformed to compare to a similarly normalized and transformed standard distribution to assess compliance with their prescribed dose. In another embodiment, the mathematically normalized and transformed standard distribution is obtained from a body of collected Urine test results.

Some limitations of the data included in the models include upper and lower bounds for urinary creatinine, urine pH, and specific gravity of the urine sample. These limitations are taken into account because these three tests (creatinine, pH, and specific gravity) are used as standards to assess the integrity and validity of urine specimen in workplace programs (Bush 2008). These limitations are another method used to ensure that “invalid” data is not utilized in the model development process.

As stated in aforementioned embodiments, the creatinine concentration is crucial for normalizing buprenorphine urine data. In addition to including the creatinine concentration, it was important to exclude patients whose creatinine concentration fall outside the U.S. Mandatory Guidelines for Federal Workplace Drug testing programs (15 to 400 mg/dL) (Bush 2008). For the purpose of developing the buprenorphine urine model only data corresponding to patients with creatinine concentrations 20 to 400 mg/dL are considered.

Specific gravity, although not considered as one of the factors in the normalization or transformation of the buprenorphine urine data, is utilized as a limiting factor for the data that is acceptable for use in the normalization and transformation process to obtain the buprenorphine urine curve. As stipulated in the Tietz Clinical Guide to Laboratory Tests, the specific gravity of a random urine sample is expected to be between 1.002 and 1.030. Values outside this range raise “red flags” with values greater than 1.030 indicating a high probability that other substances have been added to the urine sample while values below 1.002 indicate a great likelihood of urine sample dilution with water (Wu 2006). Data corresponding to patients whose urine specific gravity values fall below 1.002 or greater than 1.030 were excluded from the model.

The pH of patients' urine sample is used as a transforming factor for the buprenorphine urine models corresponding to Equation 1 and Equation 2. According to the U.S. Mandatory Guideline for Federal Workplace Drug testing programs the acceptable pH range for a “valid” randomly collected urine sample is between 4.5 and 8.0 (Bush 2008). Consequently, for all our buprenorphine urine models data is excluded for patients whose pH is less than 4.5 or greater than 8.5.

There are specific combinations of creatinine concentration, specific gravity, and urine pH that are indicators of diluted, substituted, and adulterated urine samples. Diluted urine samples usually have creatinine concentrations less than 20 mg/dL but greater than or equal to 2 mg/dL while having specific gravity less than 1.003 but greater than 1.001. Substituted urine samples usually have creatinine concentrations less than 2 mg/dL and specific gravity less than or equal to 1.001 or greater than or equal to 1.020. Adulterated urine samples usually have pH values less than 3.0 or greater than or equal to 11 (Federal Register 2004). Hence, the aforementioned cutoffs for creatinine, specific gravity, and pH ensure that all diluted, substituted, or adulterated samples are excluded from the developed model.

Furthermore, the developed model can be fine-tuned to account for different chemical compositions and pharmacological routes of administration which are taken by patients. Three of the key contributing factors that make this data extraction and separation process possible are the creatinine concentration, specific gravity, and pH. Using the aforementioned limitations for creatinine concentration, specific gravity, and pH to exclude “invalid” data result in a “best” fit of the data to a mathematical normal distribution.

In the above description, various methods have been described. It will be apparent to one of ordinary skill in the art that each of these methods may be implemented, in whole or in part, by software, hardware, and/or firmware. If implemented, in whole or in part, by software, the software may be stored on and executed by a tangible medium such as a CD-ROM, a floppy disk, a hard drive, a digital versatile disk (DVD), a read-only memory (ROM), etc.

### EXAMPLES

A summary description of the sample populations used to develop the models that are referred to in other embodiments as the buprenorphine models, alprazolam models, hydrocodone, oxycodone, and oxazepam models are described in other embodiments.

The Buprenorphine models were developed using a large batch of UDT patient data collected over the period of two years. The data was cleaned as detailed below and the resulting information used to develop the model only considered patients who tested positive for buprenorphine and were prescribed the drug. Furthermore, to ensure all the information required to adequately develop the models was available, only patients who had demographic information (patient age, patient weight, patient height) and relevant sample validity test information (sample pH and sample creatinine level), as well as drug dosage and positive urine drug concentration were included in the model. The absence or presence of illicit drugs in the patient urine test was initially considered but was found to have no significant effect on the model. Consequently, all patients were included in the model regardless of whether their illicit drug test was positive or negative. Patients whose pH and creatinine levels suggested that the sample might have been adulterated, substituted, or diluted were excluded from sample population. The total of these steps refers to as cleaning these data to afford use in preparing the model (distribution).

The resulting population used to simulate the buprenorphine models consisted of approximately 113,000 independent individual patient results of which 52% were females and 48% were males. The average age of patient included in the model was 36 years old with an average lean body weight of 55 kg. The average daily dosage of buprenorphine taken by patients included in this model was 9 mg and their average urine drug concentration was 290 ng/mL. The Urine drug concentration of buprenorphine in this model is the total buprenorphine concentration; i.e., the sum of the buprenorphine concentration and the buprenorphine glucuronide concentration (converted to buprenorphine equivalents) but could just as easily have been focused on the buprenorphine concentration only derived from methods similar to those used to detect both the glucuronide and the buprenorphine.

The alprazolam models were developed using a large batch of UDT patient data collected over a period of several years. The data was cleaned as detailed below and the resulting information used to develop the model only considered patients who tested positive for alprazolam and were prescribed the drug. Furthermore, to ensure all the information required to adequately develop the models was available, only patients who had demographic information (patient age, patient weight, patient height,) and relevant sample validity test information (sample pH and sample creatinine level), as well as drug dosage and positive urine drug concentration were included in the model. Patients whose pH and creatinine levels suggested that the sample might have been adulterated, substituted, or diluted were excluded from sample population.

The resulting population used to simulate the alprazolam models consisted of approximately 280,000 independent individual patient results of which 60% were females and 40% were males. The average age of patient included in the model was 48 years old with an average lean body weight of 55 kg. The average daily dosage of alprazolam taken by patients included in this model was 3 mg and their average urine drug concentration was 375 ng/mL.

The hydrocodone models were developed using a large batch of UDT patient data collected over a period of several years. The data were manipulated as detailed below and the resulting information used to develop the model. Only considered patients who tested positive for hydrocodone and were prescribed the drug were considered. Furthermore, to ensure all the information required to adequately develop the models was available, only patients who had demographic information (patient age, patient weight, patient height,) and relevant sample validity test information (sample pH and sample creatinine level), as well as drug dosage and positive urine drug concentration were included in the model. Patients whose pH and creatinine levels suggested that the sample might have been adulterated, substituted, or diluted were excluded from sample population.

The resulting population used to simulate the hydrocodone models consisted of approximately 100,000 independent individual patient results of which 57% were females and 43% were males. The average age of patient included in the model was 54 years old with an average lean body weight of 55 kg. The average daily dosage of hydrocodone taken by patients included in this model was 30 mg and their average urine drug concentration was 1784 ng/m L.

The oxycodone models were developed using a large batch of UDT patient data collected over a period of several years. The data was cleaned as detailed below and the resulting information used to develop the model only considered patients who tested positive for oxycodone and were prescribed the drug. Furthermore, to ensure all the information required to adequately develop the models was available, only patients who had demographic information (patient age, patient weight, patient height,) and relevant sample validity test information (sample pH and sample creatinine level), as well as drug dosage and positive urine drug concentration were included in the model. Patients whose pH and creatinine levels suggested that the sample might have been adulterated, substituted, or diluted were excluded from sample population.

The resulting population used to simulate the oxycodone models consisted of approximately 47,000 independent individual patient results of which 55% were females and 45% were males. The average age of patient included in the model was 53 years old with an average lean body weight of 56 kg. The average daily dosage of oxycodone taken by patients included in this model was 36 mg and their average urine drug concentration was 2120 ng/m L.

The oxazepam models were developed using a large batch of UDT patient data collected over a period of several years. The data were manipulated as detailed below and the resulting information used to develop the model. Only patients who tested positive for oxazepam and were prescribed diazepam and temazepam were considered. Furthermore, to ensure all the information required to adequately develop the models was available, only patients who had demographic information (patient age, patient weight, patient height,) and relevant sample validity test information (sample pH and sample creatinine level), as well as drug dosage and positive urine drug concentration were included in the model. Patients whose pH and creatinine levels suggested that the sample might have been adulterated, substituted, or diluted were excluded from sample population.

The resulting population used to simulate the oxazepam models consisted of approximately 207,000 independent individual patient results of which 56% were females and 44% were males. The average age of patient included in the model was 51 years old with an average lean body weight of 55 kg. The patients considered were primarily prescribed diazepam and temazepam. Average daily dosage taken by patients included in this model was 23 mg and their average oxazepam urine drug concentration was 1819 ng/m L.

The following examples are for illustrative purposes only and are not to be construed as limiting the scope of the present disclosure in any respect whatsoever.

### Example 1

A male subject with an age of 48 years, 25 days (48.07 years), a weight of 205 lbs, and height of 69 inches is prescribed an 8 mg daily dose of buprenorphine.

Then urine from the subject is tested. The concentration of the primary metabolite also referred to as the parent drug (e.g., buprenorphine) is 29 ng/ml. The corresponding sample fluid pH and sample fluid creatinine concentration were 5.2 and 66.2 mg/dL respectively. These values are within normal ranges so the data were processed.

Therefore, the normalized and transformed drug concentration is determined as follows using Equation 1:

$${NORM}_{{D}_{CONC}} = ln ( \frac{{P}_{MET} * LBW * pH * Age}{{D}_{DOSE} * CREAT} )$$

where LBW is calculated using Equation 6.

The standardized normal distribution value is determined using Equation 1-A:

$${NORM}_{STD ( A )} = \frac{( {NORM}_{{D}_{CONC}} - {μ}_{A} )}{{σ}_{A}}$$

The value of LBW can be determined as follows:

$$LBW = fact\_a * weight - fact\_b * {( \frac{weight}{100 * height} )}^{2} \qquad ( 6 )$$

where fact_a equals 1.1 for men and 1.07 for women and fact_b equals 128 for men and 148 for women. Weight is the subject weight measured in kg and height is the subject height in m. Hence,

$$LBW = 1.1 * ( \frac{205}{2.2} ) kg - 128 * {( \frac{( \frac{205}{2.2} ) kg}{( 100 * \frac{69}{39.37} ) m} )}^{2} = 66.317 kg$$

This leads to

$${NORM}_{{D}_{CONC}} = ln ( \frac{\frac{( 2.9 \times {10}^{- 8} ) kg}{L} * 66.317 kg * 5.2 * 48.07 years}{( 8 \times {10}^{- 6} ) kg * \frac{( 6.62 \times {10}^{- 4} ) kg}{L}} )$$

$$and$$

$${NORM}_{STD ( A )} = \frac{( {NORM}_{{D}_{CONC}} - {μ}_{A} )}{{σ}_{A}} = - 0.03$$

This patient falls within the −1 standard deviation of the model described using Equation 1 and Equation 1-A. Thus, this model would predict that this patient is compliant within +/−2 standard deviations compared to a normalized and transformed standard distribution and even more correctly, just to the left of 0 standard deviations compared to a normalized and transformed standard distribution. This patient closely resembles the population mean of “0”.

### Example 2

A female subject with an age of 19 years, 343 days (19.94 years), a weight of 114 lbs, and height of 63 inches is prescribed a 8 mg daily dose of buprenorphine.

Then urine from the subject is tested. The concentration of the primary metabolite also referred to as the parent drug (e.g., buprenorphine) is 285 ng/ml. The corresponding sample fluid pH and sample fluid creatinine concentration were 5.9 and 188 respectively.

Therefore, the normalized drug concentration is determined as follows using Equation 1:

$${NORM}_{{D}_{CONC}} = ln ( \frac{{P}_{MET} * L B W * pH * Age}{{D}_{DOSE} * CREAT} )$$

where LBW is calculated using Equation 6.
The standardized normal distribution value is determined using Equation 1-A:

$${NORM}_{STD ( A )} = \frac{( {NORM}_{{D}_{CONC}} - {μ}_{A} )}{{σ}_{A}}$$

The value of LBW can be determined as follows:

$$L B W = fact\_a * weight - fact\_b * {( \frac{weight}{100 * height} )}^{2} \qquad ( 6 )$$

where fact_a equals 1.1 for men and 1.07 for women and fact_b equals 128 for men and 148 for women. Weight is the subject weight measured in kg and height is the subject height in m. Hence,

$$L B W = 1.07 * ( \frac{114}{2.2} ) kg - 148 * {( \frac{( \frac{114}{2.2} ) kg}{( 100 * \frac{63}{39.37} ) m} )}^{2} = 39.926 kg$$

This leads to

$${NORM}_{{D}_{CONC}} = ln ( \frac{\frac{( 2.85 \times {10}^{- 7} ) kg}{L} * 39.926 kg * 5.9 * 19.94 years}{( 8 \times {10}^{- 6} ) kg * \frac{( 1.88 \times {10}^{- 3} ) kg}{L}} )$$

$$and$$

$${NORM}_{STD ( A )} = \frac{( {NORM}_{{D}_{CONC}} - {μ}_{A} )}{{σ}_{A}} = - 0.05$$

This patient falls within 1 standard deviations of the model described using Equation 1 and Equation 1-A. Thus, this model would predict that this patient is compliant within +/−2 standard deviations compared to a transformed and normalized standard distribution and even more correctly, just to the left of 0 standard deviations compared to a transformed and normalized standard distribution. This patient closely resembles the population mean.

### Example 3

A female subject with an age of 22 years, 48 days (22.13 years), a weight of 86 lbs, and height of 62 inches is prescribed a 16 mg daily dose of buprenorphine.

Then urine from the subject is tested. The concentration of the primary metabolite also referred to as the parent drug (e.g., buprenorphine) is 11 ng/ml. The corresponding sample fluid pH and sample fluid creatinine concentration were 7.9 and 166.4 respectively.

Therefore, the normalized drug concentration is determined as follows using Equation 1:

$${NORM}_{{D}_{CONC}} = ln ( \frac{{P}_{MET} * L B W * pH * Age}{{D}_{DOSE} * CREAT} )$$

where LBW is calculated using Equation 6.
The standardized normal distribution value is determined using Equation 1-A:

$${NORM}_{STD ( A )} = \frac{( {NORM}_{{D}_{CONC}} - {μ}_{A} )}{{σ}_{A}}$$

The value of LBW can be determined as follows:

$$L B W = fact\_a * weight - fact\_b * {( \frac{weight}{100 * height} )}^{2} \qquad ( 6 )$$

where fact_a equals 1.1 for men and 1.07 for women and fact_b equals 128 for Men and 148 for women. Weight is the subject weight measured in kg and height is the subject height in m. Hence,

$$L B W = 1.07 * ( \frac{86}{2.2} ) kg - 148 * {( \frac{( \frac{86}{2.2} ) kg}{( 100 * \frac{62}{39.37} ) m} )}^{2} = 32.71 kg$$

This leads to

$${NORM}_{{D}_{CONC}} = ln ( \frac{\frac{( 1.1 \times {10}^{- 8} ) kg}{L} * 32.71 kg * 7.9 * 22.13 years}{( 8 \times {10}^{- 6} ) kg * \frac{( 1.66 \times {10}^{- 3} ) kg}{L}} )$$

$$and$$

$${NORM}_{STD ( A )} = \frac{( {NORM}_{{D}_{CONC}} - {μ}_{A} )}{{σ}_{A}} = - 2.89$$

This patient falls outside −2 standard deviations of the model described using Equation 1 and Equation 1-A. Thus, this model would predict that this patient is potentially non-compliant compared to a transformed and normalized standard distribution.

### Example 4

A female subject with an age of 40 years, 200 days (40.55 years), a weight of 358 lbs, and height of 65 inches is prescribed a 4 mg daily dose of buprenorphine.

Then urine from the subject is tested. The concentration of the primary metabolite also referred to as the parent drug (e.g., buprenorphine) is 1200 ng/ml. The corresponding sample fluid pH and sample fluid creatinine concentration were 6.8 and 201.7 respectively.

Therefore, the normalized drug concentration is determined as follows using Equation 1:

$${NORM}_{{D}_{CONC}} = ln ( \frac{{P}_{MET} * L B W * pH * Age}{{D}_{DOSE} * CREAT} )$$

where LBW is calculated using Equation 6.
The standardized normal distribution value is determined using Equation 1-A:

$${NORM}_{STD ( A )} = \frac{( {NORM}_{{D}_{CONC}} - {μ}_{A} )}{{σ}_{A}}$$

The value of LBW can be determined as follows:

$$L B W = fact\_a * weight - fact\_b * {( \frac{weight}{100 * height} )}^{2} \qquad ( 6 )$$

where fact_a equals 1.1 for men and 1.07 for women and fact_b equals 128 for men and 148 for women. Weight is the subject weight measured in kg and height is the subject height in m. Hence,

$$L B W = 1.07 * ( \frac{358}{2.2} ) kg - 148 * {( \frac{( \frac{358}{2.2} ) kg}{( 100 * \frac{65}{39.37} ) m} )}^{2} = 30.34 kg$$

This leads to

$${NORM}_{{D}_{CONC}} = ln ( \frac{\frac{( 1.2 \times {10}^{- 6} ) kg}{L} * 30.34 kg * 6.8 * 40.55 years}{( 8 \times {10}^{- 6} ) kg * \frac{( 2.02 \times {10}^{- 3} ) kg}{L}} )$$

$$and$$

$${NORM}_{STD ( A )} = \frac{( {NORM}_{{D}_{CONC}} - {μ}_{A} )}{{σ}_{A}} = 2.02$$

This patient falls just outside the +2 standard deviations of the model described using Equation 1 and Equation 1-A. Thus, this model would predict that this patient is potentially non-compliant compared to a transformed and normalized standard distribution.

### Example 5

### Test of a Population of 50 Buprenorphine Patient Samples

The results (drug concentration of the primary metabolite, sample fluid pH, and sample fluid creatinine concentration), demographic information (gender, weight, height, and age), and the prescribed dosage of buprenorphine for fifty randomly selected patients—not included in the patient population used to design the models—were used to assess the validity and robustness of the models. The corresponding data is presented in Table 1. Of the patients considered in this sample 46% were females and 54% were males. The average age of patients considered in the sample set was 36 years old with an average lean body weight of 56 kg. The average daily dosage of buprenorphine taken by patients included in this model was 20 mg and their average urine drug concentration was 289 ng/m L.

TABLE 1
Drug concentrations, sample pH and creatinine concentration,
demographic information (gender, weight, height, and age), and the prescribed
dosage of buprenorphine for the sample patient population.

| Sample | | | Creatinine | Weight | Height | Age | Dose | Buprenorphine |
|---|---|---|---|---|---|---|---|---|
| Patient # | Gender | pH | (mg/dL) | (lbs) | (inches) | (yrs) | (mg) | (ng/mL) |
| 1 | M | 7.4 | 112.4 | 200 | 71 | 28.68 | 24 | 175 |
| 2 | F | 5.9 | 241.8 | 149 | 67 | 43.21 | 24 | 22 |
| 3 | F | 6.8 | 104.5 | 143 | 63 | 42.93 | 24 | 97 |
| 4 | M | 6.4 | 119.3 | 199 | 72 | 62.94 | 24 | 324 |
| 5 | F | 5.3 | 59.4 | 132 | 66 | 55.81 | 24 | 150 |
| 6 | M | 6.4 | 256.1 | 189 | 72 | 34.02 | 24 | 133 |
| 7 | F | 6.9 | 101.1 | 175 | 64 | 27.27 | 16 | 480 |
| 8 | M | 8.5 | 113.9 | 200 | 72 | 60.80 | 24 | 1768 |
| 9 | M | 7.7 | 176.4 | 178 | 70 | 27.06 | 24 | 942 |
| 10 | M | 6.2 | 210.0 | 230 | 71 | 29.26 | 24 | 47 |
| 11 | F | 8.4 | 189.5 | 132 | 64 | 23.01 | 24 | 50 |
| 12 | F | 8.4 | 36.6 | 130 | 61 | 38.44 | 24 | 51 |
| 13 | M | 6.7 | 53.2 | 168 | 68 | 41.70 | 24 | 40 |
| 14 | F | 7.7 | 55.2 | 130 | 64 | 33.86 | 24 | 119 |
| 15 | F | 6.0 | 115.2 | 154 | 64 | 37.70 | 24 | 15 |
| 16 | M | 5.8 | 146.5 | 190 | 71 | 38.49 | 24 | 332 |
| 17 | M | 6.9 | 197.6 | 181 | 65 | 35.68 | 24 | 112 |
| 18 | M | 8.2 | 55.6 | 150 | 68 | 34.01 | 24 | 46 |
| 19 | F | 7.5 | 101.3 | 135 | 63 | 50.12 | 24 | 429 |
| 20 | M | 5.3 | 63.5 | 210 | 72 | 27.63 | 24 | 799 |
| 21 | M | 6.0 | 85.2 | 180 | 71 | 34.31 | 24 | 114 |
| 22 | M | 6.1 | 99.3 | 132 | 64 | 37.21 | 24 | 111 |
| 23 | F | 8.3 | 169.0 | 175 | 70 | 35.66 | 24 | 326 |
| 24 | F | 5.7 | 237.7 | 146 | 67 | 21.46 | 24 | 1465 |
| 25 | F | 7.7 | 222.6 | 152 | 66 | 37.12 | 24 | 204 |
| 26 | M | 5.0 | 325.6 | 201 | 72 | 29.89 | 24 | 834 |
| 27 | M | 6.0 | 194.4 | 178 | 69 | 32.83 | 24 | 1433 |
| 28 | F | 8.0 | 155.0 | 159 | 69 | 29.99 | 6 | 74 |
| 29 | F | 5.6 | 50.5 | 111 | 62 | 29.03 | 12 | 605 |
| 30 | M | 6.3 | 153.7 | 173 | 73 | 25.28 | 4 | 63 |
| 31 | M | 6.3 | 226.0 | 158 | 75 | 25.99 | 24 | 206 |
| 32 | F | 6.0 | 197.3 | 198 | 68 | 28.67 | 16 | 308 |
| 33 | F | 5.7 | 142.3 | 182 | 60 | 42.12 | 2 | 16 |
| 34 | M | 6.8 | 146.6 | 225 | 75 | 31.95 | 12 | 38 |
| 35 | F | 6.5 | 127.6 | 155 | 63 | 26.53 | 8 | 59 |
| 36 | F | 7.6 | 52.3 | 120 | 61 | 21.08 | 24 | 24 |
| 37 | M | 6.7 | 148.9 | 224 | 73 | 63.11 | 8 | 115 |
| 38 | M | 6.2 | 45.7 | 181 | 69 | 61.83 | 2 | 45 |
| 39 | M | 7.5 | 16.5 | 150 | 72 | 32.15 | 24 | 34 |
| 40 | F | 8.3 | 80.3 | 146 | 68 | 30.78 | 16 | 15 |
| 41 | M | 8.1 | 74.1 | 200 | 72 | 42.36 | 24 | 63 |
| 42 | F | 5.8 | 189.1 | 135 | 65 | 29.63 | 24 | 134 |
| 43 | M | 7.8 | 101.0 | 203 | 72 | 33.25 | 24 | 216 |
| 44 | M | 7.9 | 90.6 | 170 | 70 | 50.11 | 24 | 83 |
| 45 | M | 6.5 | 166.6 | 190 | 71 | 31.42 | 24 | 748 |
| 46 | F | 6.0 | 212.9 | 110 | 69 | 31.14 | 24 | 494 |
| 47 | F | 5.8 | 258.3 | 141 | 63 | 26.93 | 16 | 85 |
| 48 | M | 8.9 | 169.6 | 191 | 73 | 44.45 | 24 | 312 |
| 49 | F | 6.9 | 104.7 | 138 | 65 | 30.99 | 24 | 72 |
| 50 | M | 5.9 | 185.5 | 152 | 73 | 31.03 | 16 | 37 |

The normalized, transformed, and standardized drug concentrations for all patients were calculated using Equation 1 and Equation 1-A, or Equation 2 and Equation 2-A, Equation 3 and Equation 3-A, or Equation 4 and Equation 4-A, or Equation 5 and Equation 5-A following the calculation of LBW, according to Equations 6 detailed in another embodiment. The calculated results for Equation 1-A, Equation 2-A, Equation 3-A, Equation 4-A, and Equation 5-A are presented in Table 2. The standard normal distribution results are presented in Table 2 and a description of whether the result was within +/−1 standard deviation, +/−2 standard deviations, or outside the range is presented in Table 3. For patient results within +/−1 standard deviation, these patients are very likely to be in compliance with their regimen. For patient results within +/−2 standard deviations, these patients are likely to be in compliance with their regimen. Patient results that fall outside the range—with the value of the standard normalized drug concentration greater than +/−2 standard deviations—are possibly non-compliant with their regimen or may have some condition not considered by the model hence causing them to not fall within at least the 95% range of the model (e.g., Rapid or absence of metabolic genetic machinery (CYP2D6)).

TABLE 2
Results for the normalized, transformed, and
standardized drug concentrations determined from
Equation 1-A, Equation 2-A, Equation 3-A,
Equation 4-A, or Equation 5-A for
50 patients prescribed Buprenorphine.

| Sample | | | | | |
|---|---|---|---|---|---|
| Patient | Equation | Equation | Equation | Equation | Equation |
| # | 1-A | 2-A | 3-A | 4-A | 5-A |
| 1 | −0.01 | 0.15 | 0.01 | −0.18 | 0.26 |
| 2 | −2.35 | −2.59 | −2.57 | −2.50 | −2.26 |
| 3 | −0.48 | −0.66 | −0.75 | −0.60 | −0.20 |
| 4 | 0.93 | 0.49 | 0.47 | 0.28 | 0.75 |
| 5 | 0.31 | −0.06 | 0.07 | 0.22 | 0.69 |
| 6 | −0.87 | −0.87 | −0.91 | −1.08 | −0.71 |
| 7 | 0.84 | 1.07 | 1.00 | 1.07 | 1.24 |
| 8 | 2.50 | 2.12 | 1.89 | 1.71 | 2.29 |
| 9 | 0.88 | 1.11 | 0.95 | 0.83 | 1.35 |
| 10 | −1.61 | −1.51 | −1.53 | −1.77 | −1.46 |
| 11 | −1.81 | −1.53 | −1.79 | −1.63 | −1.31 |
| 12 | −0.15 | −0.23 | −0.48 | −0.27 | 0.16 |
| 13 | −0.47 | −0.63 | −0.70 | −0.77 | −0.39 |
| 14 | 0.06 | 0.09 | −0.08 | 0.09 | 0.55 |
| 15 | −2.18 | −2.31 | −2.31 | −2.21 | −1.94 |
| 16 | 0.30 | 0.23 | 0.29 | 0.13 | 0.59 |
| 17 | −0.80 | −0.84 | −0.93 | −1.00 | −0.64 |
| 18 | −0.45 | −0.45 | −0.68 | −0.69 | −0.30 |
| 19 | 0.88 | 0.62 | 0.47 | 0.64 | 1.14 |
| 20 | 1.36 | 1.59 | 1.74 | 1.54 | 2.11 |
| 21 | −0.20 | −0.19 | −0.17 | −0.30 | 0.12 |
| 22 | −0.48 | −0.54 | −0.53 | −0.45 | −0.04 |
| 23 | 0.26 | 0.25 | 0.02 | 0.00 | 0.45 |
| 24 | 0.37 | 0.78 | 0.86 | 0.95 | 1.48 |
| 25 | −0.45 | −0.51 | −0.69 | −0.61 | −0.21 |
| 26 | 0.11 | 0.24 | 0.42 | 0.23 | 0.70 |
| 27 | 1.08 | 1.16 | 1.20 | 1.10 | 1.63 |
| 28 | 0.04 | 0.17 | −0.04 | −0.01 | −0.79 |
| 29 | 1.49 | 1.68 | 1.79 | 2.06 | 2.06 |
| 30 | 0.07 | 0.33 | 0.33 | 0.20 | −0.92 |
| 31 | −0.72 | −0.50 | −0.52 | −0.62 | −0.22 |
| 32 | 0.00 | 0.16 | 0.19 | 0.16 | 0.26 |
| 33 | −0.35 | −0.51 | −0.44 | −0.29 | −2.07 |
| 34 | −0.77 | −0.72 | −0.80 | −1.08 | −1.33 |
| 35 | −0.55 | −0.35 | −0.39 | −0.27 | −0.82 |
| 36 | −1.60 | −1.24 | −1.42 | −1.18 | −0.82 |
| 37 | 0.90 | 0.45 | 0.40 | 0.15 | −0.36 |
| 38 | 1.98 | 1.57 | 1.59 | 1.48 | −0.15 |
| 39 | 0.18 | 0.25 | 0.10 | 0.05 | 0.51 |
| 40 | −1.46 | −1.40 | −1.65 | −1.58 | −1.62 |
| 41 | −0.10 | −0.26 | −0.48 | −0.67 | −0.28 |
| 42 | −1.11 | −1.01 | −0.96 | −0.82 | −0.44 |
| 43 | 0.41 | 0.46 | 0.28 | 0.08 | 0.54 |
| 44 | −0.02 | −0.31 | −0.51 | −0.61 | −0.21 |
| 45 | 0.77 | 0.87 | 0.85 | 0.69 | 1.20 |
| 46 | −0.18 | −0.10 | −0.07 | 0.15 | 0.61 |
| 47 | −1.47 | −1.31 | −1.27 | −1.12 | −1.12 |
| 48 | 0.61 | 0.43 | 0.14 | −0.04 | 0.41 |
| 49 | −0.95 | −0.88 | −0.98 | −0.85 | −0.47 |
| 50 | −1.54 | −1.49 | −1.46 | −1.53 | −1.56 |

TABLE 3
Range of the results for the normalized, transformed,
and standardized drug concentrations determined
from Equation 1-A, Equation 2-A, Equation 3-A,
Equation 4-A, or Equation 5-A for
buprenorphine displayed in Table 2.

| Sample | Equation 1 | Equation 2 | Equation 3 | Equation 4 | Equation 5 |
|---|---|---|---|---|---|
| Patient # | Result | Result | Result | Result | Result |
| 1 | Within +/− 1 | Within +/− 1 | Within +/− 1 | Within +/− 1 | Within +/− 1 |
| | Std | Std | Std | Std | Std |
| 2 | Outside the | Outside the | Outside the | Outside the | Outside the |
| | Range | Range | Range | Range | Range |
| 3 | Within +/− 1 | Within +/− 1 | Within +/− 1 | Within +/− 1 | Within +/− 1 |
| | Std | Std | Std | Std | Std |
| 4 | Within +/− 1 | Within +/− 1 | Within +/− 1 | Within +/− 1 | Within +/− 1 |
| | Std | Std | Std | Std | Std |
| 5 | Within +/− 1 | Within +/− 1 | Within +/− 1 | Within +/− 1 | Within +/− 1 |
| | Std | Std | Std | Std | Std |
| 6 | Within +/− 1 | Within +/− 1 | Within +/− 1 | Within +/− 2 | Within +/− 1 |
| | Std | Std | Std | Std | Std |
| 7 | Within +/− 1 | Within +/− 2 | Within +/− 1 | Within +/− 2 | Within +/− 2 |
| | Std | Std | Std | Std | Std |
| 8 | Outside the | Outside the | Within +/− 2 | Within +/− 2 | Outside the |
| | Range | Range | Std | Std | Range |
| 9 | Within +/− 1 | Within +/− 2 | Within +/− 1 | Within +/− 1 | Within +/− 2 |
| | Std | Std | Std | Std | Std |
| 10 | Within +/− 2 | Within +/− 2 | Within +/− 2 | Within +/− 2 | Within +/− 2 |
| | Std | Std | Std | Std | Std |
| 11 | Within +/− 2 | Within +/− 2 | Within +/− 2 | Within +/− 2 | Within +/− 2 |
| | Std | Std | Std | Std | Std |
| 12 | Within +/− 1 | Within +/− 1 | Within +/− 1 | Within +/− 1 | Within +/− 1 |
| | Std | Std | Std | Std | Std |
| 13 | Within +/− 1 | Within +/− 1 | Within +/− 1 | Within +/− 1 | Within +/− 1 |
| | Std | Std | Std | Std | Std |
| 14 | Within +/− 1 | Within +/− 1 | Within +/− 1 | Within +/− 1 | Within +/− 1 |
| | Std | Std | Std | Std | Std |
| 15 | Outside the | Outside the | Outside the | Outside the | Within +/− 2 |
| | Range | Range | Range | Range | Std |
| 16 | Within +/− 1 | Within +/− 1 | Within +/− 1 | Within +/− 1 | Within +/− 1 |
| | Std | Std | Std | Std | Std |
| 17 | Within +/− 1 | Within +/− 1 | Within +/− 1 | Within +/− 2 | Within +/− 1 |
| | Std | Std | Std | Std | Std |
| 18 | Within +/− 1 | Within +/− 1 | Within +/− 1 | Within +/− 1 | Within +/− 1 |
| | Std | Std | Std | Std | Std |
| 19 | Within +/− 1 | Within +/− 1 | Within +/− 1 | Within +/− 1 | Within +/− 2 |
| | Std | Std | Std | Std | Std |
| 20 | Within +/− 2 | Within +/− 2 | Within +/− 2 | Within +/− 2 | Outside the |
| | Std | Std | Std | Std | Range |
| 21 | Within +/− 1 | Within +/− 1 | Within +/− 1 | Within +/− 1 | Within +/− 1 |
| | Std | Std | Std | Std | Std |
| 22 | Within +/− 1 | Within +/− 1 | Within +/− 1 | Within +/− 1 | Within +/− 1 |
| | Std | Std | Std | Std | Std |
| 23 | Within +/− 1 | Within +/− 1 | Within +/− 1 | Within +/− 1 | Within +/− 1 |
| | Std | Std | Std | Std | Std |
| 24 | Within +/− 1 | Within +/− 1 | Within +/− 1 | Within +/− 1 | Within +/− 2 |
| | Std | Std | Std | Std | Std |
| 25 | Within +/− 1 | Within +/− 1 | Within +/− 1 | Within +/− 1 | Within +/− 1 |
| | Std | Std | Std | Std | Std |
| 26 | Within +/− 1 | Within +/− 1 | Within +/− 1 | Within +/− 1 | Within +/− 1 |
| | Std | Std | Std | Std | Std |
| 27 | Within +/− 2 | Within +/− 2 | Within +/− 2 | Within +/− 2 | Within +/− 2 |
| | Std | Std | Std | Std | Std |
| 28 | Within +/− 1 | Within +/− 1 | Within +/− 1 | Within +/− 1 | Within +/− 1 |
| | Std | Std | Std | Std | Std |
| 29 | Within +/− 2 | Within +/− 2 | Within +/− 2 | Outside the | Outside the |
| | Std | Std | Std | Range | Range |
| 30 | Within +/− 1 | Within +/− 1 | Within +/− 1 | Within +/− 1 | Within +/− 1 |
| | Std | Std | Std | Std | Std |
| 31 | Within +/− 1 | Within +/− 1 | Within +/− 1 | Within +/− 1 | Within +/− 1 |
| | Std | Std | Std | Std | Std |
| 32 | Within +/− 1 | Within +/− 1 | Within +/− 1 | Within +/− 1 | Within +/− 1 |
| | Std | Std | Std | Std | Std |
| 33 | Within +/− 1 | Within +/− 1 | Within +/− 1 | Within +/− 1 | Outside the |
| | Std | Std | Std | Std | Range |
| 34 | Within +/− 1 | Within +/− 1 | Within +/− 1 | Within +/− 2 | Within +/− 2 |
| | Std | Std | Std | Std | Std |
| 35 | Within +/− 1 | Within +/− 1 | Within +/− 1 | Within +/− 1 | Within +/− 1 |
| | Std | Std | Std | Std | Std |
| 36 | Within +/− 2 | Within +/− 2 | Within +/− 2 | Within +/− 2 | Within +/− 1 |
| | Std | Std | Std | Std | Std |
| 37 | Within +/− 1 | Within +/− 1 | Within +/− 1 | Within +/− 1 | Within +/− 1 |
| | Std | Std | Std | Std | Std |
| 38 | Within +/− 2 | Within +/− 2 | Within +/− 2 | Within +/− 2 | Within +/− 1 |
| | Std | Std | Std | Std | Std |
| 39 | Within +/− 1 | Within +/− 1 | Within +/− 1 | Within +/− 1 | Within +/− 1 |
| | Std | Std | Std | Std | Std |
| 40 | Within +/− 2 | Within +/− 2 | Within +/− 2 | Within +/− 2 | Within +/− 2 |
| | Std | Std | Std | Std | Std |
| 41 | Within +/− 1 | Within +/− 1 | Within +/− 1 | Within +/− 1 | Within +/− 1 |
| | Std | Std | Std | Std | Std |
| 42 | Within +/− 2 | Within +/− 2 | Within +/− 1 | Within +/− 1 | Within +/− 1 |
| | Std | Std | Std | Std | Std |
| 43 | Within +/− 1 | Within +/− 1 | Within +/− 1 | Within +/− 1 | Within +/− 1 |
| | Std | Std | Std | Std | Std |
| 44 | Within +/− 1 | Within +/− 1 | Within +/− 1 | Within +/− 1 | Within +/− 1 |
| | Std | Std | Std | Std | Std |
| 45 | Within +/− 1 | Within +/− 1 | Within +/− 1 | Within +/− 1 | Within +/− 2 |
| | Std | Std | Std | Std | Std |
| 46 | Within +/− 1 | Within +/− 1 | Within +/− 1 | Within +/− 1 | Within +/− 1 |
| | Std | Std | Std | Std | Std |
| 47 | Within +/− 2 | Within +/− 2 | Within +/− 2 | Within +/− 2 | Within +/− 2 |
| | Std | Std | Std | Std | Std |
| 48 | Within +/− 1 | Within +/− 1 | Within +/− 1 | Within +/− 1 | Within +/− 1 |
| | Std | Std | Std | Std | Std |
| 49 | Within +/− 1 | Within +/− 1 | Within +/− 1 | Within +/− 1 | Within +/− 1 |
| | Std | Std | Std | Std | Std |
| 50 | Within +/− 2 | Within +/− 2 | Within +/− 2 | Within +/− 2 | Within +/− 2 |
| | Std | Std | Std | Std | Std |

Using Equation 1-A, Equation 2-A, and Equation 3-A the data closely approximates the expected normal distribution pattern with approximately 72% falling within +/−1 standard deviation (˜68%) for Equation 1 and approximately 68% falling within +/−1 standard deviation (˜68%) for Equation 2 and Equation 3, 94% falling within +/−2 standard deviations (˜95%) and 6% falling outside the +/−2 standard deviation range (˜5%). These models are very similar except Equation 2 does not account for the subject age while model 3 does not account for patient age and the sample fluid pH.

Using Equation 4 the expected normal distribution is slightly over estimated within the +/−1 standard deviation region with approximately 74% falling within +/−1 standard deviation (˜68%) for Equation 4, while 96% falling within +/−2 standard deviations (˜95%) and 4% falling outside the +/−2 standard deviation range (˜5%).

Equation 5-A is the least accurate of all the models tested, 64% of the patients fall within +/−1 standard deviation, 90% falling within +/−2 standard deviations and 10% fall outside the +/−2 standard deviation range. If we examine the parameters in Equation 5, there is no consideration of any patient specific data other than creatinine levels. This could lead to the lower predictive accuracy of the model detailed in Equation 5 and Equation 5-A in another embodiment. The use of LBW (lean body weight) in the equations 1, 2, and 3 generally leads to better model agreement with statistical expectations for a Gaussian distribution. LBW is calculated as disclosed in equation 6 using patient specific parameters and fundamentally differs from equations 4 and 5 by employing these patient specific parameters to more accurately reflect the distribution of “normal” results with buprenorphine.

Equation 5-A deviates farthest from the expected normal distribution and does not account for any patient specific parameters. Upon examination of Equation 1 and Equation 1-A, Equation 2 and Equation 2-A, Equation 3 and Equation 3-A, Equation 4 and Equation 4-A, and Equation 5 and Equation 5-A which were tested against a population of 50 patients who were prescribed buprenorphine in one of the forms detailed in other embodiments. For the model which corresponds to Equation 5 and Equation 5-A, 10% of the tested population fall outside the +/−2 standard deviation range, while models which correspond to Equation 1 and Equation 1-A, Equation 2 and Equation 2-A, and Equation 4 and Equation 4-A have 6% of the tested population fall outside the +/−2, and Equation 3 and Equation 3-A have 1% of the tested population fall outside the +/−2 standard which are closest to the expected form an standard normal distribution.

The models corresponding to Equation 1-A, Equation 2-A, Equation 3-A, and equation 4-A will adequately be able to predict the population of patients who are compliant with their regimen versus those who are potentially non-compliant or who may have conditions that may influence the effective drug absorption and or metabolism.

Comparing the models corresponding to Equation 1-A, Equation 2-A, Equation 3-A, and Equation 4-A, Equation 5-A reveals that the model correspond to Equation 5-A would present a potential non-compliant rate that is 50% greater than an ideal standardized normal distribution, 40% higher than models corresponding to Equation 1-A, Equation 2-A, and Equation 4-A, and 60% higher than the model which corresponds to Equation 3-A. In the case of Equation 5 since patient specific parameters are not accounted for, several patients who are compliant with their regimen but may be slightly underweight, overweight, or otherwise can fall outside the +/−2 standard range and be deemed as potentially non-compliant. On the other hand, some patient who would be deemed potentially compliant by the other models corresponding to Equation 1-A, Equation 2-A, Equation 3-A, and Equation 4-A that account for patient specific parameters can appear to be potentially complaint using Equation 5.

Equation 4-A adequately predicts the population that falls outside +/−2 standard deviations which is the resultant focus and covers the intended utility of any appropriate model. The model corresponding to Equation 4-A will be able to adequately predict the population of patients who are compliant with their regimen versus those who are potentially non-compliant or who may have conditions that may influence the effective drug absorption and or metabolism.

### Example 6

### Model Validity Testing

To further examine the validity of the models tested and described in Example 5 a large population of patients were tested using the same strategy. In Example 5, we detailed patient specific parameters and results for a statistically significant population of 50 randomly selected patients who were prescribed buprenorphine. In this Example 6, the model validity testing method used in Example 5 was extended to a larger population size. This model validity test included buprenorphine testing data for patients collected over a five month period. This validity test included some small modifications to the validity test described in Example 5. Example 5 only considered patients for which all patient-specific parameters were available, including: the results (drug concentration of the primary metabolite, sample fluid pH, and sample fluid creatinine concentration), demographic information (gender, weight, height, and age), and the prescribed dosage of Buprenorphine for patients. Example 6 considered all patients who were prescribed buprenorphine, regardless of the availability of patient-specific data. In a realistic reporting setting, all patients would require a normalized, transformed, and standardized normal value to be compared with the theoretical model; however, if these patient specific parameters are not available—e.g., they are undisclosed or not recorded—the patient normalized, transformed, and standardized result would be reported as “cannot be assessed”. Of the patients considered in this sample, 53% were females and 47% were males. The average age of patients considered in the sample set was 37 years old, with an average lean body weight of 56 kg. The average daily dosage of buprenorphine taken by patients included in this model was 17 mg, and their average urine drug concentration (of buprenorphine and buprenorphine equivalents of buprenorphine glucuronide) was 980 ng/mL (many patients with values above the upper limit of quantitation were considered).

Specific conditions and limitations to the models include: once a patient has a buprenorphine prescription their data will be analyzed even if demographic information (gender, weight, height, and age) was missing, and the prescribed dosage information was missing. Furthermore, even patients whose sample validity testing results (sample fluid pH, and sample fluid creatinine concentration) suggests that the sample has been substituted or adulterated were included. For the normalization and transformation process, if all the required data was not included the result returned was annotated “cannot be assessed” and this patient was not given a normalized, transformed, and standardized value. Once all the required patient specific data was available, a normalized and transformed value was returned and the patient result was described as being within +/−1 standard deviations, +/−2 standard deviations, or outside the range.

TABLE 4
Summary of patients' compliance or non-compliance results for model
validity test conducted using a large patient population of
approximately 34,000 patients (raw data presented).

| | Equation 1 | Equation 2 | Equation 3 | Equation 4 | Equation 5 |
|---|---|---|---|---|---|
| Analysis | Result | Result | Result | Result | Result |
| Total Patients | 34266 | | | | |
| Within +/− 1 | 18745 | 18763 | 18743 | 21793 | 22508 |
| Std | | | | | |
| Within +/− 2 | 24279 | 24275 | 24260 | 28419 | 30128 |
| Std | | | | | |
| Outside the | 1492 | 1496 | 1511 | 1885 | 2185 |
| Range | | | | | |
| Could not be | 8495 | 8495 | 8495 | 3962 | 1953 |
| assessed | | | | | |
| (Missing | | | | | |
| required | | | | | |
| patient | | | | | |
| information) | | | | | |

Table 4 shows a summary of the total number of patients included in this model validity test, the number of patients who fell within each compliance range, along with the number of patients who could not be assessed because the required patient specific data was not available. The total number of patients in table 4 accounts for all patients considered in the large validity test sample set. These patients were all prescribed buprenorphine and were tested over a five month period. Patients who tested negative—below the cutoff of the buprenorphine testing method (10 ng/mL) drug concentrations of the primary metabolite are recorded as 0 ng/mL—did not obtain a normalized and transformed value and were all annotated as “cannot be assessed”.

Table 5 provides evidence that Equation 5 is the least specific of all the models tested, since 100% of the patients who tested positive for buprenorphine could be assessed, only patients who tested negative for buprenorphine “could not be assessed”. This is because no patient specific information is required for this normalization and transformation process, hence once a test result is provided potential patient compliance can be determined using the model that corresponds to Equation 5. However, it should be noted that Equation 5 also produces the least accuracy with 8.5% of the tested patient population falling outside the compliance range. Models that correspond to Equation 1, Equation 2, Equation 3, and Equation 4 result in approximately 5.2 to 5.8% of the patients assessed falling outside the compliance range which correspond to the theoretical and expected value of 5% of patients falling outside +/−2 standard deviations. It can be noted that Equation 5 has approximately 55% more patients being deemed as potentially non-compliance when compared to Equation 1, Equation 2, Equation 3, and Equation 4.

TABLE 5
Summary percentage of patients who fall within different compliance
ranges produced by the model validity test that was conducted using a
large patient population of approximately 34000 patients.

| | Equation 1 | Equation 2 | Equation 3 | Equation 4 | Equation 5 |
|---|---|---|---|---|---|
| Analysis | Result | Result | Result | Result | Result |
| Within +/− 1 | 73.2 | 73.3 | 73.6 | 72.9 | 65.0 |
| Std | | | | | |
| Within +/− 2 | 94.8 | 94.5 | 94.6 | 94.2 | 91.5 |
| Std | | | | | |
| Outside the | 5.2 | 5.5 | 5.4 | 5.8 | 8.5 |
| Range | | | | | |
| Could not be | 24.8 | 24.8 | 24.8 | 11.6 | 5.7 |
| assessed | | | | | |
| (Missing | | | | | |
| required | | | | | |
| patient | | | | | |
| information) | | | | | |

TABLE 6
Summary of patients included in model validity test whose buprenorphine
concentrations are beyond the upper limit (10,000 ng/mL)

| | Equation 1 | Equation 3 | Equation 3 | Equation 4 | Equation 5 |
|---|---|---|---|---|---|
| Analysis | Result | Result | Result | Result | Result |
| Total Patients | 637 test results above the upper limit of quantitation | | | | |
| above the | 73.3 | | | | |
| upper limit | 73.6 | | | | |
| Quantitation | 72.9 | | | | |
| | 65.0 | | | | |
| Outside the | 441 | 441 | 441 | 553 | 637 |
| Range | | | | | |
| Percentage | 69% | 69% | 69% | 87% | 100% |
| Outside the | | | | | |
| Range | | | | | |
| Percentage | 31% | 31% | 31% | 13% | 0% |
| compliant | | | | | |

Table 6 provides further evidence that the consideration of patient specific data has an effect on the strength of the model to determine potential patient compliance especially if drug concentrations fall beyond the upper limit of quantitation for the LC/MSMS test. Equation 5, which excludes patient specific parameters, would determine that all patients who have drug concentrations above the upper limit are potentially non-complaint. However, Equation 4 accounts for the dosage of the buprenorphine that patients have been prescribed. Equation 4 determines that if patients are taking their drugs as prescribed approximately 13% of the patients with drug concentrations beyond the upper limit are potentially compliant. Equation 3, Equation 2, and Equation 1, accounting for patient lean body weight, patient urine specimen pH, drug dosage prescribed, and patient age estimate that approximately 31% of the patients with drug concentrations beyond the upper limit are actually potentially compliant.

### Example 7

### Test of a Population of 50 Alprazolam Patient Samples

The results (drug concentration of the primary metabolite, sample fluid pH, and sample fluid creatinine concentration), demographic information (gender, weight, height, and age), and the prescribed dosage of alprazolam for fifty randomly selected patients—not included in the patient population used to design the models—were used to assess the validity and robustness of the models. The corresponding data is presented in Table 7. Of the patients considered in this sample 70% were females and 30% were males. The average age of patients considered in the sample set was 53 years old with an average lean body weight of 53 kg. The average daily dosage of alprazolam taken by patients included in this model was 2 mg and their average urine drug concentration was 155 ng/m L.

TABLE 7
Drug concentrations, sample pH and creatinine concentration,
demographic information (gender, weight, height, and age), and the prescribed
dosage of alprazolam for the sample patient population.

| Sample | | | Creatinine | Weight | Height | Age | Dose | Alprazolam |
|---|---|---|---|---|---|---|---|---|
| Patient # | Gender | pH | (mg/dL) | (lbs) | (inches) | (yrs) | (mg) | (ng/mL) |
| 1 | F | 6.2 | 60.2 | 109 | 60 | 66.80 | 3 | 43 |
| 2 | F | 5.9 | 52 | 190 | 63 | 41.36 | 3 | 130 |
| 3 | F | 5.9 | 88.8 | 135 | 63 | 55.28 | 1 | 303 |
| 4 | F | 5.8 | 196.6 | 174 | 69 | 48.44 | 1 | 20 |
| 5 | F | 7.3 | 49.5 | 137 | 62 | 51.32 | 1 | 152 |
| 6 | F | 8.0 | 193.2 | 194 | 65 | 44.48 | 3 | 1680 |
| 7 | F | 7.3 | 32.2 | 163 | 63 | 52.10 | 3 | 22 |
| 8 | M | 6.0 | 63.7 | 208 | 69 | 73.49 | 2 | 40 |
| 9 | F | 7.3 | 60.3 | 139 | 67 | 50.54 | 1 | 72 |
| 10 | M | 7.8 | 107.0 | 229 | 75 | 71.73 | 0.25 | 79 |
| 11 | F | 7.0 | 83.9 | 115 | 64 | 46.62 | 2 | 100 |
| 12 | F | 6.0 | 208.9 | 176 | 70 | 51.26 | 3 | 110 |
| 13 | F | 8.5 | 108.7 | 280 | 68 | 38.29 | 3 | 181 |
| 14 | M | 5.6 | 82.5 | 150 | 76 | 62.95 | 1 | 51 |
| 15 | M | 4.9 | 22.4 | 245 | 70 | 56.50 | 1 | 36 |
| 16 | F | 8.0 | 19.5 | 158 | 60 | 49.22 | 3 | 178 |
| 17 | F | 7.2 | 130.4 | 235 | 66 | 54.17 | 3 | 261 |
| 18 | M | 6.2 | 262.7 | 307 | 71 | 51.81 | 3 | 107 |
| 19 | F | 6.5 | 230.2 | 114 | 63 | 54.18 | 6 | 153 |
| 20 | F | 6.4 | 10.6 | 132 | 63 | 35.90 | 3 | 22 |
| 21 | F | 6.6 | 111.7 | 171 | 62 | 37.10 | 3 | 94 |
| 22 | M | 5.9 | 122.4 | 176 | 70 | 87.15 | 0.25 | 28 |
| 23 | F | 8.1 | 18.3 | 126 | 62 | 43.49 | 3 | 69 |
| 24 | M | 5.2 | 40.9 | 191 | 69 | 56.13 | 3 | 109 |
| 25 | F | 5.9 | 337.8 | 180 | 65 | 57.56 | 1.5 | 190 |
| 26 | F | 5.9 | 252.3 | 171 | 70 | 53.80 | 3 | 203 |
| 27 | F | 5.7 | 78.3 | 150 | 70 | 47.19 | 1 | 61 |
| 28 | F | 7.4 | 39.6 | 104 | 62 | 65.03 | 3 | 146 |
| 29 | M | 7.3 | 59.4 | 176 | 70 | 53.48 | 4 | 339 |
| 30 | M | 5.2 | 147.2 | 237 | 72 | 48.10 | 1.5 | 104 |
| 31 | F | 6.4 | 22.0 | 165 | 64 | 38.96 | 1 | 74 |
| 32 | F | 7.0 | 188.6 | 156 | 63 | 57.37 | 1.5 | 80 |
| 33 | M | 6.6 | 61.1 | 209 | 73 | 68.79 | 1.5 | 37 |
| 34 | F | 7.3 | 113.5 | 143 | 64 | 49.89 | 1.5 | 99 |
| 35 | F | 8.3 | 171.6 | 106 | 64 | 55.81 | 3 | 21 |
| 36 | M | 7.1 | 113.3 | 178 | 68 | 56.71 | 1.5 | 52 |
| 37 | M | 5.8 | 78.4 | 190 | 68 | 58.51 | 2 | 250 |
| 38 | F | 7.0 | 18.2 | 111 | 62 | 45.76 | 3 | 47 |
| 39 | F | 6.5 | 115.5 | 147 | 63 | 62.86 | 2 | 302 |
| 40 | F | 7.5 | 238.4 | 223 | 65 | 62.13 | 3 | 58 |
| 41 | F | 6.3 | 135.5 | 190 | 63 | 52.81 | 1.5 | 104 |
| 42 | M | 5.9 | 17.8 | 158 | 65 | 39.50 | 4 | 94 |
| 43 | F | 7.1 | 15.3 | 135 | 62 | 47.39 | 0.25 | 97 |
| 44 | M | 5.3 | 129.0 | 226 | 73 | 41.19 | 3 | 377 |
| 45 | F | 5.2 | 246.0 | 160 | 67 | 47.96 | 2 | 205 |
| 46 | F | 7.1 | 55.6 | 254 | 58 | 51.26 | 0.5 | 27 |
| 47 | F | 7.1 | 15.9 | 160 | 64 | 61.46 | 1 | 64 |
| 48 | F | 5.6 | 124.5 | 205 | 66 | 53.55 | 2 | 40 |
| 49 | F | 6.9 | 179.9 | 161 | 66 | 52.16 | 3 | 556 |
| 50 | M | 6.9 | 62.6 | 288 | 73 | 64.87 | 1.5 | 97 |

The normalized, transformed, and standardized drug concentrations for all patients were calculated using Equation 1 and Equation 1-A, or Equation 2 and Equation 2-A, Equation 3 and Equation 3-A, or Equation 4 and Equation 4-A, or Equation 5 and Equation 5-A following the calculation of LBW, according to Equations 6 detailed in another embodiment. The calculated results for Equation 1-A, Equation 2-A, Equation 3-A, Equation 4-A, and Equation 5-A are presented in Table 7. The standard normal distribution results are presented in Table 8 and a description of whether the result was within +/−1 standard deviation, +/−2 standard deviations, or outside the range is presented in Table 9. For patient results within +/−1 standard deviation, these patients are very likely to be in compliance with their regimen. For patient results within +/−2 standard deviations, these patients are likely to be in compliance with their regimen. Patient results that fall outside the range—with the value of the standard normalized drug concentration greater than +/−2 standard deviations—are possibly non-compliant with their regimen or may have some condition not considered by the model hence causing them to not fall within at least the 95% range of the model (e.g., rapid or absence of metabolic genetic machinery (CYP2D6)).

TABLE 8
Results for the normalized, transformed, and
standardized drug concentrations determined from
Equation 1-A, Equation 2-A, Equation 3-A,
Equation 4-A, or Equation 5-A for
50 patients prescribed alprazolam.

| Sample | | | | | |
|---|---|---|---|---|---|
| Patient | Equation | Equation | Equation | Equation | Equation |
| # | 1-A | 2-A | 3-A | 4-A | 5-A |
| 1 | −1.16 | −1.51 | −1.55 | −1.22 | −1.10 |
| 2 | −0.28 | −0.18 | −0.14 | −0.07 | 0.13 |
| 3 | 1.10 | 0.99 | 1.05 | 1.23 | 0.43 |
| 4 | −1.91 | −2.00 | −1.98 | −2.00 | −3.02 |
| 5 | 1.12 | 1.08 | 0.94 | 1.13 | 0.33 |
| 6 | 1.19 | 1.27 | 1.05 | 1.08 | 1.35 |
| 7 | −1.06 | −1.19 | −1.37 | −1.26 | −1.15 |
| 8 | −0.36 | −0.77 | −0.76 | −0.96 | −1.23 |
| 9 | 0.35 | 0.30 | 0.15 | 0.26 | −0.60 |
| 10 | 1.93 | 1.61 | 1.42 | 1.09 | −1.07 |
| 11 | −0.49 | −0.50 | −0.63 | −0.37 | −0.60 |
| 12 | −1.34 | −1.46 | −1.46 | −1.50 | −1.40 |
| 13 | −0.28 | −0.10 | −0.40 | −0.44 | −0.27 |
| 14 | −0.07 | −0.33 | −0.25 | −0.34 | −1.25 |
| 15 | 0.74 | 0.59 | 0.81 | 0.54 | −0.31 |
| 16 | 1.17 | 1.16 | 0.94 | 1.12 | 1.40 |
| 17 | 0.01 | −0.11 | −0.26 | −0.27 | −0.09 |
| 18 | −1.24 | −1.37 | −1.40 | −1.73 | −1.66 |
| 19 | −1.91 | −2.10 | −2.19 | −1.92 | −1.18 |
| 20 | −0.61 | −0.39 | −0.43 | −0.24 | −0.06 |
| 21 | −1.27 | −1.10 | −1.18 | −1.07 | −0.94 |
| 22 | 0.65 | 0.11 | 0.16 | 0.02 | −2.22 |
| 23 | 0.25 | 0.33 | 0.08 | 0.31 | 0.53 |
| 24 | 0.16 | 0.00 | 0.16 | −0.01 | 0.19 |
| 25 | −0.66 | −0.86 | −0.84 | −0.80 | −1.34 |
| 26 | −0.95 | −1.10 | −1.09 | −1.11 | −0.99 |
| 27 | −0.22 | −0.24 | −0.17 | −0.13 | −1.02 |
| 28 | 0.41 | 0.12 | −0.04 | 0.29 | 0.51 |
| 29 | 0.80 | 0.71 | 0.56 | 0.43 | 0.94 |
| 30 | −0.41 | −0.44 | −0.29 | −0.59 | −1.12 |
| 31 | 0.95 | 1.14 | 1.13 | 1.21 | 0.42 |
| 32 | −0.84 | −1.05 | −1.18 | −1.06 | −1.62 |
| 33 | −0.06 | −0.40 | −0.47 | −0.73 | −1.27 |
| 34 | −0.31 | −0.38 | −0.54 | −0.40 | −0.91 |
| 35 | −2.58 | −2.81 | −3.14 | −2.84 | −2.84 |
| 36 | −0.54 | −0.72 | −0.87 | −0.99 | −1.54 |
| 37 | 0.79 | 0.61 | 0.68 | 0.53 | 0.37 |
| 38 | −0.23 | −0.21 | −0.33 | −0.04 | 0.16 |
| 39 | 0.49 | 0.25 | 0.20 | 0.35 | 0.17 |
| 40 | −1.70 | −2.00 | −2.22 | −2.21 | −2.16 |
| 41 | −0.44 | −0.56 | −0.59 | −0.51 | −1.03 |
| 42 | 0.17 | 0.33 | 0.38 | 0.36 | 0.86 |
| 43 | 2.87 | 2.95 | 2.87 | 3.07 | 1.04 |
| 44 | 0.11 | 0.23 | 0.37 | 0.08 | 0.28 |
| 45 | −0.85 | −0.89 | −0.75 | −0.70 | −0.95 |
| 46 | −0.16 | −0.24 | −0.38 | 0.07 | −1.48 |
| 47 | 1.58 | 1.39 | 1.28 | 1.38 | 0.60 |
| 48 | −1.47 | −1.63 | −1.57 | −1.58 | −1.89 |
| 49 | 0.26 | 0.17 | 0.07 | 0.13 | 0.34 |
| 50 | 0.86 | 0.60 | 0.50 | 0.13 | −0.34 |

TABLE 9
Range of the results for the normalized, transformed, and standardized
drug concentrations determined from Equation 1-A, Equation 2-A,
Equation 3-A, Equation 4-A, or Equation 5-A for alprazolam.

| Sample | Equation 1 | Equation 2 | Equation 3 | Equation 4 | Equation 5 |
|---|---|---|---|---|---|
| Patient # | Result | Result | Result | Result | Result |
| 1 | Within +/− 2 | Within +/− 2 | Within +/− 2 | Within +/− 2 | Within +/− 2 |
| | Std | Std | Std | Std | Std |
| 2 | Within +/− 1 | Within +/− 1 | Within +/− 1 | Within +/− 1 | Within +/− 1 |
| | Std | Std | Std | Std | Std |
| 3 | Within +/− 2 | Within +/− 1 | Within +/− 2 | Within +/− 2 | Within +/− 1 |
| | Std | Std | Std | Std | Std |
| 4 | Within +/− 2 | Outside the | Within +/− 2 | Within +/− 2 | Outside the |
| | Std | Range | Std | Std | Range |
| 5 | Within +/− 2 | Within +/− 2 | Within +/− 1 | Within +/− 2 | Within +/− 1 |
| | Std | Std | Std | Std | Std |
| 6 | Within +/− 2 | Within +/− 2 | Within +/− 2 | Within +/− 2 | Within +/− 2 |
| | Std | Std | Std | Std | Std |
| 7 | Within +/− 2 | Within +/− 2 | Within +/− 2 | Within +/− 2 | Within +/− 2 |
| | Std | Std | Std | Std | Std |
| 8 | Within +/− 1 | Within +/− 1 | Within +/− 1 | Within +/− 1 | Within +/− 2 |
| | Std | Std | Std | Std | Std |
| 9 | Within +/− 1 | Within +/− 1 | Within +/− 1 | Within +/− 1 | Within +/− 1 |
| | Std | Std | Std | Std | Std |
| 10 | Within +/− 2 | Within +/− 2 | Within +/− 2 | Within +/− 2 | Within +/− 2 |
| | Std | Std | Std | Std | Std |
| 11 | Within +/− 1 | Within +/− 1 | Within +/− 1 | Within +/− 1 | Within +/− 1 |
| | Std | Std | Std | Std | Std |
| 12 | Within +/− 2 | Within +/− 2 | Within +/− 2 | Within +/− 2 | Within +/− 2 |
| | Std | Std | Std | Std | Std |
| 13 | Within +/− 1 | Within +/− 1 | Within +/− 1 | Within +/− 1 | Within +/− 1 |
| | Std | Std | Std | Std | Std |
| 14 | Within +/− 1 | Within +/− 1 | Within +/− 1 | Within +/− 1 | Within +/− 2 |
| | Std | Std | Std | Std | Std |
| 15 | Within +/− 1 | Within +/− 1 | Within +/− 1 | Within +/− 1 | Within +/− 1 |
| | Std | Std | Std | Std | Std |
| 16 | Within +/− 2 | Within +/− 2 | Within +/− 1 | Within +/− 2 | Within +/− 2 |
| | Std | Std | Std | Std | Std |
| 17 | Within +/− 1 | Within +/− 1 | Within +/− 1 | Within +/− 1 | Within +/− 1 |
| | Std | Std | Std | Std | Std |
| 18 | Within +/− 2 | Within +/− 2 | Within +/− 2 | Within +/− 2 | Within +/− 2 |
| | Std | Std | Std | Std | Std |
| 19 | Within +/− 2 | Outside the | Outside the | Within +/− 2 | Within +/− 2 |
| | Std | Range | Range | Std | Std |
| 20 | Within +/− 1 | Within +/− 1 | Within +/− 1 | Within +/− 1 | Within +/− 1 |
| | Std | Std | Std | Std | Std |
| 21 | Within +/− 2 | Within +/− 2 | Within +/− 2 | Within +/− 2 | Within +/− 1 |
| | Std | Std | Std | Std | Std |
| 22 | Within +/− 1 | Within +/− 1 | Within +/− 1 | Within +/− 1 | Outside the |
| | Std | Std | Std | Std | Range |
| 23 | Within +/− 1 | Within +/− 1 | Within +/− 1 | Within +/− 1 | Within +/− 1 |
| | Std | Std | Std | Std | Std |
| 24 | Within +/− 1 | Within +/− 1 | Within +/− 1 | Within +/− 1 | Within +/− 1 |
| | Std | Std | Std | Std | Std |
| 25 | Within +/− 1 | Within +/− 1 | Within +/− 1 | Within +/− 1 | Within +/− 2 |
| | Std | Std | Std | Std | Std |
| 26 | Within +/− 1 | Within +/− 2 | Within +/− 2 | Within +/− 2 | Within +/− 1 |
| | Std | Std | Std | Std | Std |
| 27 | Within +/− 1 | Within +/− 1 | Within +/− 1 | Within +/− 1 | Within +/− 2 |
| | Std | Std | Std | Std | Std |
| 28 | Within +/− 1 | Within +/− 1 | Within +/− 1 | Within +/− 1 | Within +/− 1 |
| | Std | Std | Std | Std | Std |
| 29 | Within +/− 1 | Within +/− 1 | Within +/− 1 | Within +/− 1 | Within +/− 1 |
| | Std | Std | Std | Std | Std |
| 30 | Within +/− 1 | Within +/− 1 | Within +/− 1 | Within +/− 1 | Within +/− 2 |
| | Std | Std | Std | Std | Std |
| 31 | Within +/− 1 | Within +/− 2 | Within +/− 2 | Within +/− 2 | Within +/− 1 |
| | Std | Std | Std | Std | Std |
| 32 | Within +/− 1 | Within +/− 2 | Within +/− 2 | Within +/− 2 | Within +/− 2 |
| | Std | Std | Std | Std | Std |
| 33 | Within +/− 1 | Within +/− 1 | Within +/− 1 | Within +/− 1 | Within +/− 2 |
| | Std | Std | Std | Std | Std |
| 34 | Within +/− 1 | Within +/− 1 | Within +/− 1 | Within +/− 1 | Within +/− 1 |
| | Std | Std | Std | Std | Std |
| 35 | Outside the | Outside the | Outside the | Outside the | Outside the |
| | Range | Range | Range | Range | Range |
| 36 | Within +/− 1 | Within +/− 1 | Within +/− 1 | Within +/− 1 | Within +/− 2 |
| | Std | Std | Std | Std | Std |
| 37 | Within +/− 1 | Within +/− 1 | Within +/− 1 | Within +/− 1 | Within +/− 1 |
| | Std | Std | Std | Std | Std |
| 38 | Within +/− 1 | Within +/− 1 | Within +/− 1 | Within +/− 1 | Within +/− 1 |
| | Std | Std | Std | Std | Std |
| 39 | Within +/− 1 | Within +/− 1 | Within +/− 1 | Within +/− 1 | Within +/− 1 |
| | Std | Std | Std | Std | Std |
| 40 | Within +/− 2 | Outside the | Outside the | Outside the | Outside the |
| | Std | Range | Range | Range | Range |
| 41 | Within +/− 1 | Within +/− 1 | Within +/− 1 | Within +/− 1 | Within +/− 2 |
| | Std | Std | Std | Std | Std |
| 42 | Within +/− 1 | Within +/− 1 | Within +/− 1 | Within +/− 1 | Within +/− 1 |
| | Std | Std | Std | Std | Std |
| 43 | Outside the | Outside the | Outside the | Outside the | Within +/− 2 |
| | Range | Range | Range | Range | Std |
| 44 | Within +/− 1 | Within +/− 1 | Within +/− 1 | Within +/− 1 | Within +/− 1 |
| | Std | Std | Std | Std | Std |
| 45 | Within +/− 1 | Within +/− 1 | Within +/− 1 | Within +/− 1 | Within +/− 1 |
| | Std | Std | Std | Std | Std |
| 46 | Within +/− 1 | Within +/− 1 | Within +/− 1 | Within +/− 1 | Within +/− 2 |
| | Std | Std | Std | Std | Std |
| 47 | Within +/− 2 | Within +/− 2 | Within +/− 2 | Within +/− 2 | Within +/− 1 |
| | Std | Std | Std | Std | Std |
| 48 | Within +/− 2 | Within +/− 2 | Within +/− 2 | Within +/− 2 | Within +/− 2 |
| | Std | Std | Std | Std | Std |
| 49 | Within +/− 1 | Within +/− 1 | Within +/− 1 | Within +/− 1 | Within +/− 1 |
| | Std | Std | Std | Std | Std |
| 50 | Within +/− 1 | Within +/− 1 | Within +/− 1 | Within +/− 1 | Within +/− 1 |
| | Std | Std | Std | Std | Std |

Using Equation 1-A the data closely approximates the expected normal distribution pattern with approximately 66% falling within +/−1 standard deviation (˜68%), 96% falling within +/−2 standard deviations (˜95%) and 4% falling outside the +/−2 standard deviation range (˜5%). Using Equation 2-A, Equation 3-A, Equation 4-A, Equation 5-A the data slightly deviates from the expected normal distribution pattern with approximately 62% falling within +/−1 standard deviation (˜68%), 90% falling within +/−2 standard deviations (˜95%) and 10% falling outside the +/−2 standard deviation range (˜5%) for Equation 2-A. Approximately 64% falling within +/−1 standard deviation (˜68%), 92% falling within +/−2 standard deviations (˜95%) and 8% falling outside the +/−2 standard deviation range (˜5%) for Equation 3-A. Approximately 60% falling within +/−1 standard deviation (˜68%), 94% falling within +/−2 standard deviations (˜95%) and 6% falling outside the +/−2 standard deviation range (˜5%) for Equation 4-A. Approximately 52% falling within +/−1 standard deviation (˜68%), 92% falling within +/−2 standard deviations (˜95%) and 8% falling outside the +/−2 standard deviation range (˜5%) for Equation 5-A.

All of the models considered, corresponding to Equation 1-A, Equation 2-A, Equation 3-A, Equation 4-A, and Equation 5-A, predicts the percentage of patients who would be potentially compliant (within +/−2 standard deviations of the mean) and potentially non-compliant (outside of the +/−2 standard deviations of the mean range) to a fairly accurate degree when compared to the theoretical expectation of a model that closely fits or resembles a standard normal distribution. However, upon closer examination of the spread of all the models, the model corresponding to Equation 1-A seems to be the strongest model since it most closely overlaps with the expected standard normal distribution with approximately 66% falling within +/−1 standard deviation (˜68%), 96% falling within +/−2 standard deviations (˜95%) and 4% falling outside the +/−2 standard deviation range (˜5%).

### Example 8

### Test of a Population of 50 Oxazepam Patient Samples

The results (drug concentration of the primary metabolite, sample fluid pH, and sample fluid creatinine concentration), demographic information (gender, weight, height, and age), and the prescribed dosage of parent drugs (diazepam and temazepam), which have oxazepam as a metabolite for fifty randomly selected patients—not included in the patient population used to design the models—were used to assess the validity and robustness of the models. The corresponding data is presented in Table 10. Of the patients considered in this sample 58% were females and 42% were males. The average age of patients considered in the sample set was 54 years old with an average lean body weight of 53 kg. The average daily dosage of either diazepam or temazepam taken by patients included in this model was 17 mg and their average urine drug concentration was 1096 ng/m L.

TABLE 10
Drug concentrations, sample pH and creatinine concentration,
demographic information (gender, weight, height, and age), and the prescribed
dosage of (diazepam or temazepam) for the sample patient population.

| Sample | | | Creatinine | Weight | Height | Age | Dose | Oxazepam |
|---|---|---|---|---|---|---|---|---|
| Patient # | Gender | pH | (mg/dL) | (lbs) | (inches) | (yrs) | (mg) | (ng/mL) |
| 1 | F | 7.3 | 143.4 | 201 | 64 | 62.40 | 15 | 2049 |
| 2 | M | 8.8 | 117.8 | 181 | 73 | 53.58 | 15 | 375 |
| 3 | F | 7.1 | 20.5 | 140 | 64 | 59.66 | 15 | 387 |
| 4 | M | 6.1 | 429.8 | 219 | 72 | 66.35 | 40 | 2769 |
| 5 | M | 5.1 | 394.5 | 187 | 74 | 43.31 | 30 | 4271 |
| 6 | F | 5.6 | 161.5 | 197 | 75 | 57.51 | 10 | 444 |
| 7 | F | 5.7 | 164.7 | 135 | 61 | 67.70 | 10 | 534 |
| 8 | F | 8.0 | 130.3 | 150 | 62 | 47.38 | 40 | 107 |
| 9 | M | 6.5 | 123.6 | 169 | 71 | 65.31 | 5 | 426 |
| 10 | M | 8.2 | 37.9 | 142 | 69 | 76.03 | 10 | 590 |
| 11 | F | 5.3 | 178.9 | 170 | 65 | 49.78 | 10 | 254 |
| 12 | F | 7.8 | 35.7 | 115 | 65 | 72.19 | 45 | 259 |
| 13 | F | 5.9 | 161.4 | 184 | 61 | 34.50 | 10 | 149 |
| 14 | F | 8.7 | 45.2 | 254 | 65 | 59.82 | 5 | 216 |
| 15 | F | 7.3 | 103 | 298 | 65 | 56.61 | 10 | 611 |
| 16 | F | 8.0 | 53.5 | 240 | 67 | 54.34 | 20 | 740 |
| 17 | F | 7.5 | 67.1 | 214 | 60 | 51.71 | 20 | 1470 |
| 18 | M | 6.3 | 31.4 | 285 | 74 | 38.13 | 10 | 60 |
| 19 | F | 6.8 | 20.3 | 180 | 65 | 47.75 | 15 | 25 |
| 20 | M | 5.6 | 102.5 | 176 | 66 | 48.18 | 10 | 306 |
| 21 | F | 6.7 | 19.2 | 183 | 69 | 34.61 | 10 | 935 |
| 22 | M | 6.9 | 25.2 | 155 | 66 | 56.67 | 30 | 2309 |
| 23 | F | 8.8 | 82 | 139 | 60 | 51.70 | 15 | 64 |
| 24 | M | 5.5 | 58.5 | 167 | 60 | 40.13 | 6 | 187 |
| 25 | M | 5.9 | 431.1 | 178 | 65 | 25.22 | 15 | 394 |
| 26 | M | 8.1 | 59.2 | 292 | 65 | 54.00 | 10 | 179 |
| 27 | F | 7.2 | 38.7 | 202 | 66 | 47.86 | 10 | 210 |
| 28 | F | 5.2 | 108.9 | 332 | 64 | 44.62 | 20 | 2534 |
| 29 | F | 5.5 | 97 | 146 | 61 | 59.71 | 10 | 649 |
| 30 | F | 6.1 | 117.6 | 188 | 67 | 34.96 | 20 | 3842 |
| 31 | M | 7.0 | 24 | 211 | 64 | 49.94 | 30 | 627 |
| 32 | F | 6.6 | 145 | 171 | 65 | 43.04 | 10 | 787 |
| 33 | M | 6.2 | 39.6 | 191 | 67 | 53.22 | 30 | 1606 |
| 34 | M | 5.5 | 177.6 | 203 | 64 | 48.62 | 5 | 540 |
| 35 | F | 5.9 | 44.1 | 176 | 63 | 59.72 | 20 | 176 |
| 36 | F | 6.3 | 35 | 150 | 62 | 56.78 | 10 | 411 |
| 37 | M | 5.7 | 102.2 | 150 | 66 | 62.56 | 10 | 1671 |
| 38 | M | 5.0 | 104.7 | 202 | 71 | 69.99 | 30 | 3594 |
| 39 | M | 5.0 | 300.8 | 188 | 73 | 45.48 | 10 | 2118 |
| 40 | F | 5.5 | 105.6 | 131 | 70 | 61.17 | 15 | 3062 |
| 41 | F | 7.2 | 27.2 | 158 | 65 | 67.19 | 10 | 599 |
| 42 | M | 5.1 | 35.6 | 152 | 68 | 60.49 | 10 | 214 |
| 43 | F | 7.5 | 63.5 | 128 | 63 | 58.17 | 40 | 5345 |
| 44 | F | 7.2 | 30 | 166 | 64 | 52.42 | 5 | 169 |
| 45 | F | 6.0 | 177.2 | 116 | 60 | 49.23 | 2 | 1694 |
| 46 | M | 5.8 | 252 | 177 | 68 | 49.69 | 6 | 536 |
| 47 | M | 7.0 | 61.8 | 131 | 67 | 61.69 | 30 | 842 |
| 48 | F | 5.0 | 38.9 | 170 | 66 | 61.91 | 15 | 394 |
| 49 | M | 6.6 | 47.4 | 132 | 68 | 56.52 | 20 | 1339 |
| 50 | F | 6.7 | 145.7 | 166 | 61 | 57.94 | 30 | 1708 |

The normalized, transformed, and standardized drug concentrations for all patients were calculated using Equation 1 and Equation 1-A, or Equation 2 and Equation 2-A, Equation 3 and Equation 3-A, or Equation 4 and Equation 4-A, or Equation 5 and Equation 5-A following the calculation of LBW, according to Equations 6 detailed in another embodiment. Note that inasmuch as oxazepam is the first metabolite of temazepam or diazepam the drug concentration term (Pmet) had to be adjusted to 1/Pmet. This affords the same Gaussian distribution as modelling the parent drug. The calculated results for Equation 1-A, Equation 2-A, Equation 3-A, Equation 4-A, and Equation 5-A are presented in Table 10. The standard normal distribution results are presented in Table 11 and a description of whether the result was within +/−1 standard deviation, +/−2 standard deviations, or outside the range is presented in Table 12. For patient results within +/−1 standard deviation, these patients are very likely to be in compliance with their regimen. For patient results within +/−2 standard deviations, these patients are likely to be in compliance with their regimen. Patient results that fall outside the range—with the value of the normalized drug concentration greater than +/−2 standard deviations—are possibly non-compliant with their regimen or may have some condition not considered by the model hence causing them to not fall within at least the 95% range of the model (e.g., Rapid or absence of metabolic genetic machinery (CYP2D6)).

TABLE 11
Results for the normalized, transformed, and standardized drug
concentrations determined from Equation 1-A, Equation 2-A,
Equation 3-A, Equation 4-A, or Equation 5-A for
50 patients prescribed (diazepam and
temezapam) which have oxazepam as a metabolite.

| Sample | | | | | |
|---|---|---|---|---|---|
| Patient | Equation | Equation | Equation | Equation | Equation |
| # | 1-A | 2-A | 3-A | 4-A | 5-A |
| 1 | −0.42 | −0.55 | −0.64 | −0.61 | −0.78 |
| 2 | 0.68 | 0.65 | 0.47 | 0.37 | 0.37 |
| 3 | 1.31 | 1.23 | 1.17 | 1.26 | 1.40 |
| 4 | −1.52 | −1.69 | −1.70 | −1.85 | −1.62 |
| 5 | −1.89 | −1.85 | −1.77 | −1.88 | −1.83 |
| 6 | 0.43 | 0.36 | 0.41 | 0.33 | 0.07 |
| 7 | 0.22 | 0.06 | 0.10 | 0.23 | −0.05 |
| 8 | 0.47 | 0.50 | 0.37 | 0.46 | 1.06 |
| 9 | 1.06 | 0.93 | 0.92 | 0.85 | 0.26 |
| 10 | 1.27 | 1.07 | 0.93 | 0.94 | 0.78 |
| 11 | 0.45 | 0.45 | 0.53 | 0.57 | 0.35 |
| 12 | 0.77 | 0.59 | 0.47 | 0.61 | 1.31 |
| 13 | 0.60 | 0.79 | 0.82 | 0.90 | 0.73 |
| 14 | 1.93 | 1.86 | 1.70 | 1.73 | 1.28 |
| 15 | 0.46 | 0.39 | 0.31 | 0.40 | 0.15 |
| 16 | 0.47 | 0.43 | 0.30 | 0.28 | 0.43 |
| 17 | −0.17 | −0.20 | −0.30 | −0.19 | −0.12 |
| 18 | 2.26 | 2.42 | 2.44 | 2.22 | 2.27 |
| 19 | 2.64 | 2.69 | 2.67 | 2.69 | 3.06 |
| 20 | 0.73 | 0.75 | 0.81 | 0.76 | 0.57 |
| 21 | 0.90 | 1.10 | 1.07 | 1.05 | 0.91 |
| 22 | 0.01 | −0.06 | −0.12 | −0.13 | 0.20 |
| 23 | 1.52 | 1.52 | 1.35 | 1.48 | 1.65 |
| 24 | 1.35 | 1.48 | 1.55 | 1.57 | 1.21 |
| 25 | −0.64 | −0.31 | −0.29 | −0.33 | −0.45 |
| 26 | 1.56 | 1.53 | 1.41 | 1.32 | 1.23 |
| 27 | 1.49 | 1.53 | 1.46 | 1.46 | 1.39 |
| 28 | −1.09 | −1.05 | −0.97 | −0.73 | −0.74 |
| 29 | 0.32 | 0.23 | 0.29 | 0.40 | 0.15 |
| 30 | −1.14 | −0.98 | −0.99 | −0.98 | −1.04 |
| 31 | 0.69 | 0.69 | 0.63 | 0.57 | 1.02 |
| 32 | 0.02 | 0.09 | 0.05 | 0.09 | −0.21 |
| 33 | −0.06 | −0.10 | −0.11 | −0.18 | 0.15 |
| 34 | 0.52 | 0.53 | 0.60 | 0.54 | −0.10 |
| 35 | 1.12 | 1.04 | 1.07 | 1.13 | 1.42 |
| 36 | 1.13 | 1.07 | 1.07 | 1.17 | 1.04 |
| 37 | −0.04 | −0.16 | −0.12 | −0.12 | −0.45 |
| 38 | −0.89 | −1.08 | −0.98 | −1.10 | −0.93 |
| 39 | −0.88 | −0.85 | −0.75 | −0.80 | −1.24 |
| 40 | −0.66 | −0.79 | −0.73 | −0.66 | −0.83 |
| 41 | 1.26 | 1.12 | 1.05 | 1.10 | 0.97 |
| 42 | 1.43 | 1.35 | 1.46 | 1.50 | 1.43 |
| 43 | −1.11 | −1.21 | −1.32 | −1.20 | −0.86 |
| 44 | 2.08 | 2.07 | 2.02 | 2.07 | 1.67 |
| 45 | 0.23 | 0.23 | 0.25 | 0.42 | −0.79 |
| 46 | 0.29 | 0.29 | 0.33 | 0.27 | −0.31 |
| 47 | 0.07 | −0.05 | −0.12 | −0.07 | 0.27 |
| 48 | 0.88 | 0.78 | 0.89 | 0.92 | 1.01 |
| 49 | 0.10 | 0.03 | 0.00 | 0.03 | 0.15 |
| 50 | −0.83 | −0.92 | −0.98 | −0.89 | −0.68 |

TABLE 12
Range of the results for the normalized, transformed, and
standardized drug concentrations determined from
Equation 1-A, Equation 2-A, Equation 3-A,
Equation 4-A, or Equation 5-A for oxazepam displayed in Table 12.

| Sample | Equation 1 | Equation 2 | Equation 3 | Equation 4 | Equation 5 |
|---|---|---|---|---|---|
| Patient # | Result | Result | Result | Result | Result |
| 1 | Within +/− 1 | Within +/− 1 | Within +/− 1 | Within +/− 1 | Within +/− 1 |
| | Std | Std | Std | Std | Std |
| 2 | Within +/− 1 | Within +/− 1 | Within +/− 1 | Within +/− 1 | Within +/− 1 |
| | Std | Std | Std | Std | Std |
| 3 | Within +/− 2 | Within +/− 2 | Within +/− 2 | Within +/− 2 | Within +/− 2 |
| | Std | Std | Std | Std | Std |
| 4 | Within +/− 2 | Within +/− 2 | Within +/− 2 | Within +/− 2 | Within +/− 2 |
| | Std | Std | Std | Std | Std |
| 5 | Within +/− 2 | Within +/− 2 | Within +/− 2 | Within +/− 2 | Within +/− 2 |
| | Std | Std | Std | Std | Std |
| 6 | Within +/− 1 | Within +/− 1 | Within +/− 1 | Within +/− 1 | Within +/− 1 |
| | Std | Std | Std | Std | Std |
| 7 | Within +/− 1 | Within +/− 1 | Within +/− 1 | Within +/− 1 | Within +/− 1 |
| | Std | Std | Std | Std | Std |
| 8 | Within +/− 1 | Within +/− 1 | Within +/− 1 | Within +/− 1 | Within +/− 2 |
| | Std | Std | Std | Std | Std |
| 9 | Within +/− 2 | Within +/− 1 | Within +/− 1 | Within +/− 1 | Within +/− 1 |
| | Std | Std | Std | Std | Std |
| 10 | Within +/− 2 | Within +/− 2 | Within +/− 1 | Within +/− 1 | Within +/− 1 |
| | Std | Std | Std | Std | Std |
| 11 | Within +/− 1 | Within +/− 1 | Within +/− 1 | Within +/− 1 | Within +/− 1 |
| | Std | Std | Std | Std | Std |
| 12 | Within +/− 1 | Within +/− 1 | Within +/− 1 | Within +/− 1 | Within +/− 2 |
| | Std | Std | Std | Std | Std |
| 13 | Within +/− 1 | Within +/− 1 | Within +/− 1 | Within +/− 1 | Within +/− 1 |
| | Std | Std | Std | Std | Std |
| 14 | Within +/− 2 | Within +/− 2 | Within +/− 2 | Within +/− 2 | Within +/− 2 |
| | Std | Std | Std | Std | Std |
| 15 | Within +/− 1 | Within +/− 1 | Within +/− 1 | Within +/− 1 | Within +/− 1 |
| | Std | Std | Std | Std | Std |
| 16 | Within +/− 1 | Within +/− 1 | Within +/− 1 | Within +/− 1 | Within +/− 1 |
| | Std | Std | Std | Std | Std |
| 17 | Within +/− 1 | Within +/− 1 | Within +/− 1 | Within +/− 1 | Within +/− 1 |
| | Std | Std | Std | Std | Std |
| 18 | Outside the | Outside the | Outside the | Outside the | Outside the |
| | Range | Range | Range | Range | Range |
| 19 | Outside the | Outside the | Outside the | Outside the | Outside the |
| | Range | Range | Range | Range | Range |
| 20 | Within +/− 1 | Within +/− 1 | Within +/− 1 | Within +/− 1 | Within +/− 1 |
| | Std | Std | Std | Std | Std |
| 21 | Within +/− 1 | Within +/− 2 | Within +/− 2 | Within +/− 2 | Within +/− 1 |
| | Std | Std | Std | Std | Std |
| 22 | Within +/− 1 | Within +/− 1 | Within +/− 1 | Within +/− 1 | Within +/− 1 |
| | Std | Std | Std | Std | Std |
| 23 | Within +/− 2 | Within +/− 2 | Within +/− 2 | Within +/− 2 | Within +/− 2 |
| | Std | Std | Std | Std | Std |
| 24 | Within +/− 2 | Within +/− 2 | Within +/− 2 | Within +/− 2 | Within +/− 2 |
| | Std | Std | Std | Std | Std |
| 25 | Within +/− 1 | Within +/− 1 | Within +/− 1 | Within +/− 1 | Within +/− 1 |
| | Std | Std | Std | Std | Std |
| 26 | Within +/− 2 | Within +/− 2 | Within +/− 2 | Within +/− 2 | Within +/− 2 |
| | Std | Std | Std | Std | Std |
| 27 | Within +/− 2 | Within +/− 2 | Within +/− 2 | Within +/− 2 | Within +/− 2 |
| | Std | Std | Std | Std | Std |
| 28 | Within +/− 2 | Within +/− 2 | Within +/− 1 | Within +/− 1 | Within +/− 1 |
| | Std | Std | Std | Std | Std |
| 29 | Within +/− 1 | Within +/− 1 | Within +/− 1 | Within +/− 1 | Within +/− 1 |
| | Std | Std | Std | Std | Std |
| 30 | Within +/− 2 | Within +/− 1 | Within +/− 1 | Within +/− 1 | Within +/− 2 |
| | Std | Std | Std | Std | Std |
| 31 | Within +/− 1 | Within +/− 1 | Within +/− 1 | Within +/− 1 | Within +/− 2 |
| | Std | Std | Std | Std | Std |
| 32 | Within +/− 1 | Within +/− 1 | Within +/− 1 | Within +/− 1 | Within +/− 1 |
| | Std | Std | Std | Std | Std |
| 33 | Within +/− 1 | Within +/− 1 | Within +/− 1 | Within +/− 1 | Within +/− 1 |
| | Std | Std | Std | Std | Std |
| 34 | Within +/− 1 | Within +/− 1 | Within +/− 1 | Within +/− 1 | Within +/− 1 |
| | Std | Std | Std | Std | Std |
| 35 | Within +/− 2 | Within +/− 2 | Within +/− 2 | Within +/− 2 | Within +/− 2 |
| | Std | Std | Std | Std | Std |
| 36 | Within +/− 2 | Within +/− 2 | Within +/− 2 | Within +/− 2 | Within +/− 2 |
| | Std | Std | Std | Std | Std |
| 37 | Within +/− 1 | Within +/− 1 | Within +/− 1 | Within +/− 1 | Within +/− 1 |
| | Std | Std | Std | Std | Std |
| 38 | Within +/− 1 | Within +/− 2 | Within +/− 1 | Within +/− 2 | Within +/− 1 |
| | Std | Std | Std | Std | Std |
| 39 | Within +/− 1 | Within +/− 1 | Within +/− 1 | Within +/− 1 | Within +/− 2 |
| | Std | Std | Std | Std | Std |
| 40 | Within +/− 1 | Within +/− 1 | Within +/− 1 | Within +/− 1 | Within +/− 1 |
| | Std | Std | Std | Std | Std |
| 41 | Within +/− 2 | Within +/− 2 | Within +/− 2 | Within +/− 2 | Within +/− 1 |
| | Std | Std | Std | Std | Std |
| 42 | Within +/− 2 | Within +/− 2 | Within +/− 2 | Within +/− 2 | Within +/− 2 |
| | Std | Std | Std | Std | Std |
| 43 | Within +/− 2 | Within +/− 2 | Within +/− 2 | Within +/− 2 | Within +/− 1 |
| | Std | Std | Std | Std | Std |
| 44 | Outside the | Outside the | Outside the | Outside the | Within +/− 2 |
| | Range | Range | Range | Range | Std |
| 45 | Within +/− 1 | Within +/− 1 | Within +/− 1 | Within +/− 1 | Within +/− 1 |
| | Std | Std | Std | Std | Std |
| 46 | Within +/− 1 | Within +/− 1 | Within +/− 1 | Within +/− 1 | Within +/− 1 |
| | Std | Std | Std | Std | Std |
| 47 | Within +/− 1 | Within +/− 1 | Within +/− 1 | Within +/− 1 | Within +/− 1 |
| | Std | Std | Std | Std | Std |
| 48 | Within +/− 1 | Within +/− 1 | Within +/− 1 | Within +/− 1 | Within +/− 2 |
| | Std | Std | Std | Std | Std |
| 49 | Within +/− 1 | Within +/− 1 | Within +/− 1 | Within +/− 1 | Within +/− 1 |
| | Std | Std | Std | Std | Std |
| 50 | Within +/− 1 | Within +/− 1 | Within +/− 1 | Within +/− 1 | Within +/− 1 |
| | Std | Std | Std | Std | Std |

All of the models considered, corresponding to Equation 1-A, Equation 2-A, Equation 3-A, Equation 4-A, and Equation 5-A, predict the percentage of patients who would be potentially compliant (within +/−2 standard deviations of the mean) and potentially non-compliant (outside of the +/−2 standard deviations of the mean range) to a fairly accurate degree when compared to the theoretical expectation of a model that closely fits or resembles a standard normal distribution. For Equation 1-A, Equation 2-A, Equation 3-A, and Equation 4-A, 94% fall within +/−2 standard deviations (˜95%) and 6% falling outside the +/−2 standard deviation range (˜5%). For Equation 5-A 96% fall within +/−2 standard deviations (˜95%) and 4% falling outside the +/−2 standard deviation range (˜5%).

### REFERENCES

1. R. C. Baselt. Disposition of toxic drugs and chemicals in man, 7^{th} edition. Chemical Toxicology Institute, Foster City, Calif., 2004, pp. 136-138.
2. Suboxone, highlights of prescribing information, Reference ID: 3496928, April 2014 Revision, http://www.suboxone.com/content/pdfs/SuboxonePl.pdf Accessed Oct. 3, 2014.
3. Butrans® highlights of prescribing information, June 2014 Revision, http://app.purduepharma.com/xmlpublishing/pi.aspx?id=b Accessed Oct. 3, 2014.
4. Center for Substance Abuse Treatment. Clinical guidelines for the use of buprenorphine in the treatment of opioid addiction. Treatment Improvement Protocol (TIP) Series 40. DHHS Publication No. (SMA) 04-3939. Rockville, Md.: Substance Abuse and Mental Health Services Administration, 2004.
5. S. L. Kacinko, H. E. Jones, R. E. Johnson, R. E. Choo and M. A. Huestis. Correlations of maternal buprenorphine dose, buprenorphine, and metabolite concentrations in meconium with neonatal outcomes. Clin. Pharmacol. Ther. 84(5):604-612. (2008).
6. National Drug Intelligence Center. Intelligence bulletin: Buprenorphine: potential for abuse. Product no. 2004-L0424-013. US Department of Justice (2004).
7. S. L. Kacinko, H. E. Jones, R. E. Johnson, R. E. Choo, M. Concheiro-Guisan and M. A. Huestis. Urinary excretion of buprenorphine, norbuprenorphine, buprenorphine-glucuronide, and norbuprenorphine-glucuronide in pregnant women receiving buprenorphine maintenance treatment. Clin. Chem. 55(6):1177-1187. (2009).
8. E. Cone, C. Gorodetzky, D. Yousefnejad, W. Buchwald, R. Johnson. The metabolism and excretion of buprenorphine in humans. Drug Metab Dispos. 12: 577-581. (1984).
9. Substance Abuse and Mental Health Services Administration. Clinical drug testing in primary care. Technical Assistance Publication (TAP) 32. HHS Publication No. (SMA) 12-4668. Rockville, Md.: Substance Abuse and Mental Health Services Administration. (2012).
10. L. R. Webster, The role of urine drug testing in chronic pain management: 2013 update. Pain Medicine News Special ed. 45-50. (2013).
11. A. R. Absalom, V. Mani, T. DeSmet et al., Pharmacokinetic models for propofol-defining and illuminating the devil in the detail. Br J Anaesth 103:26-37 (2009).
12. B. E. Cole. Recognizing and preventing medication diversion. Fam Pract Manag. 8(9): 37-41. (2001).
13. D. M. Bush, the U.S. Mandatory guidelines for federal workplace drug testing programs: current status and future considerations. Forensic Sci. Int. 174: 111-9 (2008).
14. A. H. Wu, Tietz Clinical Guide to Laboratory Tests 4^{th} Edition (2006)
15. Substance abuse and mental health services administration, department of health and human services, mandatory guidelines for federal workplace drug testing programs, Federal Register, 69:19643-73 (2004).
16. H. Leider, METHODS OF NORMALIZING MEASURED DRUG CONCENTRATIONS AND TESTING FOR POTENTIAL NON-COMPLIANCE WITH A DRUG TREATMENT REGIMEN, PCT application No. 61/792,472, Mar. 15, 2013

I/We claim:

1. A method of determining non-compliance with a prescribed drug regimen in a subject, the method comprising:
determining a prescribed daily dose of drug in a subject;
determining an age, a weight, a height, and a gender of the subject;
determining the creatinine level in urine of the subject;
measuring a concentration of a primary metabolite of the drug in urine of the subject;
determining a mathematically normalized and transformed metabolite concentration as a function of parameters comprising the creatinine concentration in the urine the concentration of the primary metabolite, the age, the weight, the gender, the body mass index, the body surface area, the lean body weight of the subject, and the prescribed dosage of the drug; and
comparing the mathematically normalized and transformed metabolite concentration to a similarly normalized and transformed standard distribution derived from a collection of urine test results of patients known to be prescribed the drug in question and who tested positive for that drug or metabolite in their urine.

2. The method of claim 1 further comprising measuring a concentration of a secondary metabolite in the urine of the subject, wherein the parameters used in determining the mathematically normalized and transformed metabolite concentration comprise the concentration of the secondary metabolite (e.g., 1/[secondary metabolite]).

3. The method of claim 1 wherein the parameters used in determining the mathematically normalized and transformed metabolite concentration consist of the creatinine, the concentration of the primary metabolite, the age, the weight, the gender, the body mass index, the body surface area, and the lean body weight of the subject and the prescribed dosage of the drug.

4. The method of claim 2 wherein the parameters used in determining the mathematically normalized and transformed metabolite concentration consist of the creatinine, the inverse of the concentration of the secondary metabolite, the age, the weight, the gender, the body mass index, the body surface area, and the lean body weight of the subject and the prescribed dosage of the drug.

5. The method of claim 1 wherein the plurality of standard deviation values are a −2 standard deviation value, a 0 standard deviation value, and a +2 standard deviation value which for a Gaussian distribution, contains approximately 95% of the total of the population in question.

6. The method of claim 1 wherein the plurality of standard deviation values are a −1 standard deviation value, a 0 standard deviation value, and a +1 standard deviation value which for a Gaussian distribution, contains approximately 68% of the total of the population in question.

7. The method of claim 6 further comprising comparing the mathematically normalized and transformed drug or metabolite concentration to the standard deviation of the standard distribution derived from a collection of urine test results of patients known to be prescribed the drug in question and who tested positive for that drug or metabolite in their urine.

8. The method of claim 7 further comprising comparing the mathematically normalized and transformed drug or metabolite concentration to the −1 standard deviation value or the 0 standard deviation or the +1 standard deviation value.

9. The method of claim 1 wherein the mathematically transformed value is based at least partially on a natural log of the measured urine concentration of the drug or metabolite.

10. The method of claim 1 wherein the mathematically normalized and transformed value is based at least partially on two or more parameters of the patient selected from weight, height, sex, prescribed daily dose, Lean Body Weight (LBW), Body Surface Area (BSA), and/or uric acid concentration.

11. The method of claim 10 wherein the mathematically normalized and transformed value determined at least partially on the natural logarithm of one or more of the given parameters of the patient including: weight, height, LBW, BSA, and the prescribed daily dose of the drug in question is compared to a similarly normalized and transformed standard distribution derived from a collection of urine test results of patients known to be prescribed the drug in question and who tested positive for that drug or metabolite in their urine.

12. The method of claim 10 wherein the mathematically normalized and transformed value determined at least partially on the natural logarithm of one or more of the given parameters of the patient including: weight, height, LBW, BSA, and the prescribed daily dose of the drug in question is compared to the standard deviation of a similarly normalized and transformed standard distribution derived from a collection of urine test results of patients known to be prescribed the drug in question and who tested positive for that drug or metabolite in their urine.

13. The method of claim 10 wherein the mathematically normalized and transformed value determined at least partially on the natural logarithm of one or more of the given parameters of the patient including: weight, height, LBW, BSA, and the prescribed daily dose of the drug in question is compared to +/−2 standard deviations of a similarly normalized and transformed standard distribution derived from a collection of urine test results of patients known to be prescribed the drug in question and who tested positive for that drug or metabolite in their urine.

14. The method of claim 1 wherein the final mathematically normalized and transformed score is based at least partially on one or more adjustment variables derived from the samples of the population.

15. The method of claim 1 wherein the drug is selected from the group consisting of controlled release buprenorphine, sub-lingual tablets of buprenorphine, topical “patches” of buprenorphine, controlled-release oxycodone, oxycodone, controlled release morphine, morphine, extended release morphine hydrocodone, methadone, and a combination of controlled-release oxycodone and oxycodone.

16. The method of claim 1 wherein the parameters consist of the lean body weight, the concentration of the primary metabolite, the age, the weight and the gender of the subject.

17. The method of claim 1 further comprising determining if the subject is compliant with a drug regimen that includes the prescribed daily dose of the drug.

18. The method of claim 1 wherein the primary metabolite is the drug.

19. The method of claim 1 wherein the drug is an opioid or an antipsychotic drug.

20. The method of claim 1 wherein the drug is selected from a benzodiazepine and/or a benzodiazepine metabolite.

21. The method of claim 1 wherein the drug is buprenorphine or marijuana.

22. The method of claim 1 wherein the drug is a chronically prescribed medication that normally demonstrates a steady state level in patients.

23. The method of claim 1 wherein the drug is an antidepressant, an anticonvulsant, methylphenidate, dexamphetamine, adderol lisdexamphetamine an amphetamine derivative or any other drug used to treat attention deficit hyperactivity disorder (ADHD) and/or the symptoms of Autism spectrum disorder (ASD)
